(19) United States
(12) Patent Application Publication
(10) Pub. No.: US 2012/0252020 A1
(43) Pub. Date: Oct. 4, 2012

(54) Screening Assay for Bladder Cancer

(75) Inventor: Anthony P. Shuber, Mendon, MA (US)

(73) Assignee: PREDICTIVE BIOSCIENCES, INC., Lexington, MA (US)

(21) Appl. No.: 13/472,339

(22) Filed: May 15, 2012

Related U.S. Application Data
(63) Continuation-in-part of application No. 13/161,074, filed on Jun. 15, 2011.
Application No. 13/161,074 is a continuation-in-part of application No. 12/034,698, filed on Feb. 21, 2008.
Application No. 12/034,698 is a continuation-in-part of application No. 11/840,777, filed on Aug. 17, 2007.
(60) Provisional application No. 60/972,507, filed on Sep. 14, 2007.

(51) Int. Cl. C12Q 1/68

(52) U.S. Cl. 435/6.11

(57) ABSTRACT

The present invention generally relates to methods of screening for cancer. Methods of the invention involve identifying a threshold parameter of a protein and of two or more nucleic acids, where the threshold parameters are indicative of the absence of cancer, conducting an assay in a sample to determine a parameter of the two or more nucleic acids and a parameter of the protein, and identifying the sample as positive for cancer if the parameters of at least one of the nucleic acids and the protein present in the sample are greater than their respective threshold parameters. In certain aspects of the invention, the nucleic acids include FGFR3, p53, TWIST1, Vimentin, and NID2. In certain aspects of the invention, the protein includes MMP2 or MMP9.

### RELATED APPLICATION

The present application is a continuation-in-part of U.S. nonprovisional patent application serial number 13/161,074, filed Jun. 15, 2011, which is a continuation-in-part of U.S. nonprovisional patent application Ser. No. 12/034,698, filed Feb. 21, 2008, which claims the benefit of and priority to U.S. provisional patent application Ser. No. 60/972,507, filed Sep. 14, 2007. The present application is also a continuation-in-part of U.S. nonprovisional patent application Ser. No. 11/840,777, filed Aug. 17, 2007. The content of each application thus listed is incorporated by reference herein in its entirety.

### FIELD OF THE INVENTION

The present invention generally relates to the detection of cancer using a combination of protein and DNA markers.

### BACKGROUND

Biomarkers are naturally occurring molecules, genes, or characteristics that can be used to monitor a physiological process or condition. Standard screening assays have been developed that use biomarkers to assess the health status of a patient and to provide insight into the patient's risk of having a particular disease or condition. Screening assays generally employ a threshold above which a patient is screened as “positive” for the indicated disease and below which the patient is screened as “negative” for the indicated disease. Those tests vary not only in accuracy, precision and reliability, but have performance characteristics, e.g., sensitivity, specificity, positive predictive value (PPV) and negative predictive value (NPV). Test sensitivity and specificity refer to the identification of patients with and without the disease, respectively. For a test to be useful, it must have high sensitivity and specificity. The PPV refers to the proportion of persons who tested positive who have the disease, and the NPV refers to the number of persons who tested negative for a disease and who do not have the disease.

One problem with those diagnostic tests used in clinical practice is that the tests are single analyte, e.g. the tests assay only a single biomarker, or the tests assay a single characteristic across multiple analytes, e.g. the tests assay only gene expression or only mutations across multiple biomarkers. By looking at only a single biomarker or single characteristic across multiple biomarkers, these tests have limited sensitivity and specificity, and thus a certain number of patients will have a result that does not allow them to be unambiguously placed into any clinical category, resulting in a high number of false positive and false negative results. This ambiguity limits the usefulness of biomarker assays for cancer diagnosis.

In many cases, invasive procedures to obtain tissue samples are often considered necessary for these diagnostic tests despite a low percentage of patients that actually are positive for the disease and the ambiguity of such tests. Bladder cancer, for example, is any of several types of malignancies associated with the bladder epithelial lining. The presence of blood in the urine (hematuria) is one of the hallmark symptoms of bladder cancer. The cause of hematuria is conventionally diagnosed using cystoscopy, an invasive procedure, in which a tube-like instrument is used to look inside the urethra and bladder. Hematuria, however, is also attributable to other many non-cancer causes, such as menstruation, vigorous exercise, infection, etc., such that the vast majority of individuals with hematuria (˜95%) who are screened by cystoscopy do not have bladder cancer. This suggests that the prevalence of cancer in this population may be lower than 5%, making screening by cystoscopy inefficient, and in a large number of cases, unnecessary. Accordingly, there is a need for a more efficient means of diagnosing cancer, including bladder cancer, which reduces the need for invasive procedures and eliminates the ambiguity associated with conventional biomarker assays.

### SUMMARY

The present invention provides methods for detecting cancer using a combination of proteins and nucleic acid biomarkers in a single multi-analyte diagnostic screening assay. Methods of the invention take advantage of the fact that multiple biomarkers may be indicative of a single cancer or disease and that certain combinations of nucleic acid and protein biomarkers in an assay result in an optimal predictive value, i.e. the combinations have increased specificity while maintaining high sensitivity. The biomarkers encompassed by the invention can be obtained through non-invasive means, such as a urine sample, and through use of single molecule sequencing, the urine-based assays achieve results with similar sensitivity as invasive tissue-based assays. Accordingly, the need for particularly inconvenient and invasive procedures such as cystoscopies is reduced while the predictive value of the assay is advantageously increased.

In certain aspects, the clinical status of a patient is determined using a single multi-analyte screening assay that combines protein and nucleic acid biomarkers to assess whether a sample is positive for cancer. The screening assay includes identifying a threshold parameters for two or more nucleic acids, in which the threshold parameters is indicative of an absence of cancer, conducting an assay in a tissue or body fluid sample in order to determine an parameter of two or more nucleic acids, determining a level of one or more proteins in the sample, and identifying the sample as positive for cancer if the parameters of one or more of the nucleic acids or levels one or more of the proteins present in the sample are greater than their respective threshold parameter.

Aspects of the invention are particularly useful in complex diagnostic assessments because the multiplex analysis of a plurality of biomarkers increases the diagnostic power and accuracy of the result. According to one aspect of the invention, a plurality of different biomarkers obtained from a sample is assessed compared to a baseline or threshold amount for the respective biomarker. Each biomarker is then assigned a binary result (e.g. a 1 or a 0) based upon whether the detected level of the biomarker exceeds a predetermined threshold. Then, a cumulative score is obtained by adding the binary results to produce a diagnostic score that is used in clinical evaluation. In one embodiment, biomarkers are weighed based upon known diagnostic criteria and/or patient history, lifestyle, symptoms, and the like. The resulting aggregate weighted score is used for clinical assessment.

Threshold parameters or values for any particular biomarker and associated cancer or disease are determined by reference to literature, standard of care criteria or may be determined empirically. In a preferred embodiment of the invention, thresholds for use in association with biomarker panels of the invention are based upon positive and negative predictive values associated with threshold parameters of the marker. In one example, markers are chosen that provide 100% negative predictive value, in other words patients having values of a sufficient number of markers (which may be only one) below assigned threshold values are not expected to have the disease for which the screen is being conducted and can unambiguously be determined not to need further intervention at that time. Conversely, threshold parameters can be set so as to achieve approximately 100% positive predictive value. In that case, a critical number of biomarker levels above that threshold are unambiguously associated with the need for further intervention. As will be apparent to the skilled artisan, positive and negative predictive values for certain biomarkers do not have to be 100%, but can be something less than that depending upon other factors, such as the patients genetic history or predisposition, overall health, the presence or absence of other markers for diseases, etc.

The biomarkers used may be any biomarkers known in the art to have a predictive value or suspected predictive value associated with a condition or conditions being diagnosed. In certain aspects, the biomarkers analyzed have a predicted or suspected predictive value associate with cancer, such as bladder cancer. When the biomarker is a nucleic acid biomarker, the assay may be used to detect presence or absence of a mutation, in which presence of the mutation is indicative of a positive result for the disease. When the biomarker is a protein biomarker, the assay measures a level of the protein in the sample, in which a level exceeding a predetermined threshold for the protein is indicative of a positive result for the disease. In other embodiments, a level below a predetermined threshold for the protein is indicative of a positive result for the disease. The biomarkers have a known standard-of-care threshold for disease diagnosis, which is easily knowable by one of skill in the art by reference to literature.

Accordingly, in one embodiment of the invention, a plurality of biomarkers is measured in a sample obtained from a patient. In one aspect, the plurality of biomarkers for an assay includes two or more nucleic acids and one or more proteins (including antibodies, enzymes, etc.). Depending on the purpose of the screening, the nucleic acids are assayed to detect parameters such as nucleic acid mutations (such as additions, deletions, translocations, single nucleotide polymorphism), loss of heterozygosity, gene expression, and methylation patterns. In the case of proteins, the protein expression level can be analyzed.

Markers chosen for multi-analyte screening assays are chosen based upon their predictive value or suspected predictive value for the condition or conditions being diagnosed. Particular markers are selected based upon various diagnostic criteria, such as suspected association with disease. The number of markers chosen is at the discretion of the user and depends upon the cumulative predictive ability of the markers and the specificity/sensitivity of individual markers in the panel. A panel of markers can be chosen to increase the effectiveness of diagnosis, prognosis, treatment response, and/or recurrence. In addition to general concerns around specificity and sensitivity, markers can also be chosen in consideration of the patient's history and lifestyle. For example, other diseases that the patient has, might have, or has had can effect the choice of biomarkers to be analyzed. Drugs that the patient has in his/her system may also affect the panel.

The invention is especially useful in screening for cancers, such as bladder cancer. For example, nucleic acids such as FGFR3, p53, TWIST1, vimentin (VIM), and NID2 are nucleic acid biomarkers associated with bladder cancers. Specifically, mutations in FGFR3 and p53, hypermethylation of TWIST1, NID2, and VIM are all indicative of a positive result for bladder cancer. See, for example, Renard et al. Eur Urol. 2010 July; 58(1):96-104. Epub 2009 Aug. 5; Fernandez et al. Research and Reports in Urology 2012:4: 17-26; Council et al. Modern Pathology (2009) 22, 639-650. These nucleic acid biomarkers typically have minimal overlap with each other and can exist as independent predictors of bladder cancer with varying levels of predictive performance. Furthermore, proteins such as MMP-2 and MMP-9 are also linked to bladder cancer. For example, biologically active MMP-2 and MMP-9 are found at higher levels and at a greater frequency in urine of bladder cancer patients than in healthy patients. See, for example, Eissa S. et al., Eur. Urol. 2007; 52(5): 1388-1396.

In light of the above, methods of the invention provide for a multi-analyte screening assay that examines at least two nucleic acids selected from the group consisting of FGFR3, pp 53, TWIST1, VIM and NID2 and at least one protein selected from the group consisting of MMP-2 and MMP-9. Because FGFR3, p53, VIM, TWIST1, and NID2 have minimal overlap and can exist as independent predictors of bladder cancer, combining these nucleic acid biomarkers into a single multi-analyte screening assay provides an increased predictive value over a single analyte assay of any one of the nucleic acid biomarkers. In addition, MMP-2 and/or MMP-9 proteins are also included in the single multi-analyte screening assay to complement nucleic acid biomarkers. The addition of MMP-2 and MMP-9 protein biomarkers increases the negative predictive value of the nucleic acid biomarker assay by positively identifying patients who do not have bladder cancer.

The nucleic acid and protein biomarkers can be detected using any method known in the art, e.g. sequencing-based technologies, array-based technologies, or both. In one aspect, nucleic acid biomarkers are analyzed using a sequencing platform and protein biomarkers are analyzed using an immunoassay, such as an ELISA assay.

In a preferred embodiment, both nucleic acid and protein biomarkers are assayed on the same analytical platform, such as a sequencing platform. In such aspect, an aptamer is added to a sample that binds to a target protein to form an aptamer/protein complex. Subsequent sequencing and detection of the aptamer represents an amount of target protein in the sample. In such embodiment, methods of the invention provide for obtaining a sample comprising two or more nucleic acids and one or more proteins, introducing an aptamer that binds to the protein in the sample, removing unbound aptamer, and conducting a single assay, wherein the assay detects both said nucleic acids and said protein by performing a sequencing reaction on the two or more nucleic acids and the aptamer. In a particular embodiment, nucleic acid biomarkers such as FGFR3, p53, TWIST1, NID2, and VIM, and protein biomarkers such as MMP-2 and MMP-9 are assayed on the same analytical platform to diagnose bladder cancer. This reduces the costs diagnosing bladder cancer by eliminating the plurality of platforms previously required to screen multiple analytes. Biomarker assays according to the invention can also be conducted on separate platforms, wherein the results are combined as taught herein to provide an overall diagnostic result. In a particular embodiment, protein biomarkers are assayed on an ELISA-based platform while genetic markers are assayed using a PCR or sequencing based platform. The results from the separate platforms are subsequently weighted accordingly and combined.

A preferred sequencing technique for the screening assay is single molecule sequencing for assaying nucleic acid biomarkers, protein biomarkers, or both.

Further aspects and features of the invention will be apparent upon inspection of the following detailed description thereof.

### DETAILED DESCRIPTION

Methods of the invention provide a sensitive and specific test for detecting and diagnosing different diseases or disorders, particularly cancer. In certain aspects, the screening assay includes identifying a threshold parameter for a protein and for two or more nucleic acids, wherein the threshold parameters are indicative of the absence of cancer, conducting an assay in a tissue or body fluid sample in order to determine a parameter for the two or more nucleic acids selected, determining a parameter for at least one or more proteins in the sample, and identifying the sample as positive for cancer if the parameters of at least one of the nucleic acids and the parameter of the protein present in the sample exceed their respective threshold parameters.

The invention allows the use of different analytes or biomarkers in a single diagnostic algorithm in order to increase predictive power. According to the invention, multiple analytes are measured and the measured outputs are converted into a single readout score or a signature that is predictive of clinical outcome. The readout can be binary (e.g., 1/0, yes/no) or can be a point on a continuum that represents a degree of risk of disease or severity or likely outcome (e.g., of treatment, recurrence, etc.). In any of these cases, the readout is correlated to predictive outcomes at a desired level of confidence. For example, upon analysis of multiple analytes, a signature can be generated based upon the pattern of results obtained for the selected panel. That signature is then correlated to clinical outcome based upon comparison to a training set with the same panel or empirically based upon prior results. The determination of individual analyte results can also be placed into a bar code format that can be structured to correlate with clinical outcome. Individual assay results can either be weighted or not and can either be normalized or not depending upon the needs of the overall result.

By way of example, one aspect of the invention provides a binary algorithm in which nucleic acid and protein measurements are made in order to provide a diagnostic readout. In this example, an assay is conducted to determine whether a mutation exists in a genomic region known to associate with cancer. For example, a single nucleotide polymorphism known to be predictive of disease onset is first determined. There are numerous means for doing this, such as single base extension assays (e.g., U.S. Pat. No. 6,566,101, incorporated by reference herein). A result indicating whether the mutation is present or not (1 or 0) is obtained. Several other DNA mutations can be measured as well and similarly assigned a binary score for disease association. As many mutation-based assays as are desired can be performed. The level of a protein or proteins known to be informative for cancer is also measured. This could be, for example, the tumor suppressor p53 protein. It is determined whether the level of that protein exceeds a threshold amount known to be indicative of the presence of disease. A binary result is also assigned to this analyte (e.g., 1 if threshold is exceeded and 0 if it is not). Finally, a quantitative RNA assay may be performed to determine the level or levels of diagnostically-relevant RNA expressed in the sample. A binary result is obtained based upon the expression levels obtained for each RNA species measured, and comparison to known disease-associated thresholds. The result of all these assays is a series of binary outcomes that form a barcode-type readout that is assigned clinical status based upon a priori determinations of disease association for the entire marker panel.

In another aspect of the invention, each of the assayed biomarkers produces a quantitative result that is also assigned a weighted value based upon how much of the analyte is present in the sample relative to a predetermined threshold for the marker. For each marker, a result above the cutoff is given a weighted positive score (in this case based upon amount present in excess of the cutoff) and those below the threshold are given a weighted negative score. The weighted scores are then assessed to provide an overall diagnostic readout.

Biomarkers chosen are immaterial to the operation of the invention as long as the marker is associated with the disease for which screening is being conducted. Exemplary biomarkers include nucleic acid biomarkers and protein biomarkers. Biomarkers used in methods of the invention are chosen based upon their predictive value or suspected predictive value for the condition or conditions being diagnosed. Particular markers are selected based upon various diagnostic criteria, such as suspected association with disease. The number of markers chosen will depend on the number of assays performed and is at the discretion of the user. Biomarkers should be chosen that cumulatively increase the specificity/sensitivity of the assay. A panel of markers can be chosen to increase the effectiveness of diagnosis, prognosis, treatment response, and/or recurrence. In addition to general concerns around specificity and sensitivity, markers can also be chosen in consideration of the patient's history and lifestyle. For example, other diseases that the patient has, might have, or has had can effect the choice of the panel of biomarkers to be analyzed. Drugs that the patient has in his/her system may also affect biomarker selection.

Threshold values for any particular biomarker and associated disease are determined by reference to literature or standard of care criteria or may be determined empirically. In certain embodiments of the invention, thresholds for use in association with biomarkers of the invention are based upon positive and negative predictive values associated with threshold levels of the marker. There are numerous methods for determining thresholds for use in the invention, including reference to standard values in the literature or associated standards of care. The precise thresholds chosen are immaterial as long as they have the desired association with diagnostic output.

The invention is applicable to diagnosis and monitoring of any disease, either in symptomatic or asymptomatic patient populations. For example, the invention can be used for diagnosis of infectious diseases, inherited diseases, and other conditions, such as disease or damage caused by drug or alcohol abuse. The invention can also be applied to assess therapeutic efficacy, potential for disease recurrence or spread (e.g. metastasis).

Methods of the invention can be used on patients known to have a disease, or can be used to screen healthy subjects on a periodic basis. Screening can be done on a regular basis (e.g., weekly, monthly, annually, or other time interval); or as a one-time event. The outcome of the analysis may be used to alter the frequency and/or type of screening, diagnostic and/or treatment protocols. Different conditions can be screened for at different time intervals and as a function of different risk factors (e.g., age, weight, gender, history of smoking, family history, genetic risks, exposure to toxins and/or carcinogens etc., or a combination thereof). The particular screening regimen and choice of markers used in connection with the invention are determined at the discretion of the physician or technician.

Biomarkers associated with diseases are shown for example in Shuber (U.S. patent application number 2009/0075266), the content of which is incorporated by reference herein in its entirety. The invention is especially useful in screening for cancer. Examples of biomarkers associated with cancer include FGFR3, matrix metalloproteinase (MMP), neutrophil gelatinase-associated lipocalin (NGAL), MMP/NGAL complex, thymosin β15, thymosin β16, collagen like gene (CLG) product, prohibitin, glutathione-S-transferase, beta-5-tubulin, ubiquitin, tropomyosin, Cyr61, cystatin B, chaperonin 10, and profilin. Examples of MMPs include, but are not limited to, MMP-2, MMP-9, MMP9/NGAL complex, MMP/TIMP complex, MMP/TIMP1 complex, ADAMTS-7 or ADAM-12, among others.

Biomarkers associated with development of breast cancer are shown in Erlander et al. (U.S. Pat. No. 7,504,214), Dai et al. (U.S. Pat. Nos. 7,514,209 and 7,171,311), Baker et al. (U.S. Pat. No. 7,056,674 and U.S. Pat. No. 7,081,340), Erlander et al. (US 2009/0092973). The contents of the patent application and each of these patents are incorporated by reference herein in their entirety. Exemplary biomarkers that have been associated with breast cancer include: ErbB2 (Her2); ESR1; BRCA1; BRCA2; p53; mdm2; cyclin1; p27; B_Catenin; BAG1; BIN1; BUB1; C20_orf1; CCNB1; CCNE2; CDC20; CDH1; CEGP1; CIAP1; cMYC; CTSL2; DKFZp586M07; DR5; EpCAM; EstR1; FOXM1; GRB7; GSTM1; GSTM3; HER2; HNRPAB; ID1; IGF1R; ITGA7; Ki_{—}67; KNSL2; LMNB1; MCM2; MELK; MMP12; MMP9; MYBL2; NEK2; NME1; NPD009; PCNA; PR; PREP; PTTG1; RPLPO; Src; STK15; STMY3; SURV; TFRC; TOP2A; and TS.

Biomarkers associated with development of cervical cancer are shown in Patel (U.S. Pat. No. 7,300,765), Pardee et al. (U.S. Pat. No. 7,153,700), Kim (U.S. Pat. No. 6,905,844), Roberts et al. (U.S. Pat. No. 6,316,208), Schlegel (US 2008/0113340), Kwok et al. (US 2008/0044828), Fisher et al. (US 2005/0260566), Sastry et al. (US 2005/0048467), Lai (US 2008/0311570) and Van Der Zee et al. (US 2009/0023137). The contents of each of the articles, patents, and patent applications are incorporated by reference herein in their entirety. Exemplary biomarkers that have been associated with cervical cancer include: SC6; SIX1; human cervical cancer 2 protooncogene (HCCR-2); p27; virus oncogene E6; virus oncogene E7; p16^{INK4A}; Mcm proteins (such as Mcm5); Cdc proteins; topoisomerase 2 alpha; PCNA; Ki-67; Cyclin E; p-53; PAI1; DAP-kinase; ESR1; APC; TIMP-3; RAR-β; CALCA; TSLC1; TIMP-2; DcR1; CUDR; DcR2; BRCA1; p15; MSH2; Rassf1A; MLH1; MGMT; SOX1; PAX1; LMX1A; NKX6-1; WT1; ONECUT1; SPAG9; and Rb (retinoblastoma) proteins.

Biomarkers associated with development of vaginal cancer are shown in Giordano (U.S. Pat. No. 5,840,506), Kruk (US 2008/0009005), Hellman et al. (Br J. Cancer. 100(8):1303-1314, 2009). The contents of each of the articles, patents, and patent applications are incorporated by reference herein in their entirety. Exemplary biomarkers that have been associated with vaginal cancer include: pRb2/p130 and Bc1-2.

Biomarkers associated with development of brain cancers (e.g., glioma, cerebellum, medulloblastoma, astrocytoma, ependymoma, glioblastoma) are shown in D'Andrea (US 2009/0081237), Murphy et al. (US 2006/0269558), Gibson et al. (US 2006/0281089), and Zetter et al. (US 2006/0160762). The contents of each of the articles and patent applications are incorporated by reference herein in their entirety. Exemplary biomarkers that have been associated with brain cancers include: epidermal growth factor receptor (EGFR); phosphorylated PKB/Akt; EGFRvIII; FANCI; Nr-CAM; antizyme inhibitor (AZI); BNIP3; and miRNA-21.

Biomarkers associated with development of renal cancer are shown in Patel (U.S. Pat. No. 7,300,765), Soyupak et al. (U.S. Pat. No. 7,482,129), Sahin et al. (U.S. Pat. No. 7,527,933), Price et al. (U.S. Pat. No. 7,229,770), Raitano (U.S. Pat. No. 7,507,541), and Becker et al. (US 2007/0292869). The contents of each of the articles, patents, and patent applications are incorporated by reference herein in their entirety. Exemplary biomarkers that have been associated with renal cancers include: SC6; 36P6D5; IMP3; serum amyloid alpha; YKL-40; SC6; and carbonic anhydrase IX (CA IX).

Biomarkers associated with development of hepatic cancers (e.g., hepatocellular carcinoma) are shown in Home et al. (U.S. Pat. No. 6,974,667), Yuan et al. (U.S. Pat. No. 6,897,018), Hanausek-Walaszek et al. (U.S. Pat. No. 5,310,653), and Liew et al. (US 2005/0152908). The contents of each of the articles, patents, and patent applications are incorporated by reference herein in their entirety. Exemplary biomarkers that have been associated with hepatic cancers include: Tetraspan NET-6 protein; collagen, type V, alpha; glypican 3; pituitary tumor-transforming gene 1 (PTTG1); Galectin 3; solute carrier family 2, member 3, or glucose transporter 3 (GLUT3); metallothionein 1L; CYP2A6; claudin 4; serine protease inhibitor, Kazal type I (SPINK1); DLC-1; AFP; HSP70; CAP2; glypican 3; glutamine synthetase; AFP; AST and CEA.

Biomarkers associated with development of gastric, gastrointestinal, and/or esophageal cancers are shown in Chang et al. (U.S. Pat. No. 7,507,532), Bae et al. (U.S. Pat. No. 7,368,255), Muramatsu et al. (U.S. Pat. No. 7,090,983), Sahin et al. (U.S. Pat. No. 7,527,933), Chow et al. (US 2008/0138806), Waldman et al. (US 2005/0100895), Goldenring (US 2008/0057514), An et al. (US 2007/0259368), Guilford et al. (US 2007/0184439), Wirtz et al. (US 2004/0018525), Filella et al. (Acta Oncol. 33(7):747-751, 1994), Waldman et al. (U.S. Pat. No. 6,767,704), and Lipkin et al. (Cancer Research, 48:235-245, 1988). The contents of each of the articles, patents, and patent applications are incorporated by reference herein in their entirety. Exemplary biomarkers that have been associated with gastric, gastrointestinal, and/or esophageal cancers include: MH15 (Hn1L); RUNX3; midkine; Chromogranin A (CHGA); Thy-1 cell surface antigen (THY1); IPO-38; CEA; CA 19.9; GroES; TAG-72; TGM3; HE4; LGALS3; IL1RN; TRIP13; FIGNL1; CRIP1; S100A4; EXOSC8; EXPI; CRCA-1; BRRN1; NELF; EREG; TMEM40; TMEM109; and guanylin cyclase C.

Biomarkers associated with development of ovarian cancer are shown in Podust et al. (U.S. Pat. No. 7,510,842), Wang (U.S. Pat. No. 7,348,142), O'Brien et al. (U.S. Pat. Nos. 7,291,462, 6,942,978, 6,316,213, 6,294,344, and 6,268,165), Ganetta (U.S. Pat. No. 7,078,180), Malinowski et al. (US 2009/0087849), Beyer et al. (US 2009/0081685), Fischer et al. (US 2009/0075307), Mansfield et al. (US 2009/0004687), Livingston et al. (US 2008/0286199), Farias-Eisner et al. (US 2008/0038754), Ahmed et al. (US 2007/0053896), Giordano (U.S. Pat. No. 5,840,506), and Tchagang et al. (Mol Cancer Ther, 7:27-37, 2008). The contents of each of the articles, patents, and patent applications are incorporated by reference herein in their entirety. Exemplary biomarkers that have been associated with ovarian cancer include: hepcidin; tumor antigen-derived gene (TADG-15); TADG-12; TADG-14; ZEB; PUMP-1; stratum corneum chymotrytic enzyme (SCCE); NES-1; μPA; PAI-2; cathepsin B; cathepsin L; ERCC5; MMP-2; pRb2/p130 gene; matrix metalloproteinase-7 (MMP-7); progesterone-associated endometrial protein (PALP); cancer antigen 125 (CAI25); CTAP3; human epididymis 4 (HL4); plasminogen activator urokinase receptor (PLAUR); MUC-1; FGF-2; cSHMT; Tbx3; utrophin; SLP1; osteopontin (SSP1); mesothelin (MSLN); SPON1; interleukin-7; folate receptor 1; and claudin 3.

Biomarkers associated with development of head-and-neck and thyroid cancers are shown in Sidransky et al. (U.S. Pat. No. 7,378,233), Skolnick et al. (U.S. Pat. No. 5,989,815), Budiman et al. (US 2009/0075265), Hasina et al. (Cancer Research, 63:555-559, 2003), Kebebew et al. (US 2008/0280302), and Ralhan (Mol Cell Proteomics, 7(6):1162-1173, 2008). The contents of each of the articles, patents, and patent applications are incorporated by reference herein in their entirety. Exemplary biomarkers that have been associated with head-and-neck and thyroid cancers include: BRAF; Multiple Tumor Suppressor (MTS); PAI-2; stratifin; YWHAZ; S100-A2; S100-A7 (psoriasin); S100-A11 (calgizarrin); prothymosin alpha (PTHA); L-lactate dehydrogenase A chain; glutathione S-transferase Pi; APC-binding protein EB1; fascin; peroxiredoxin2; carbonic anhydrase I; flavin reductase; histone H3; ECM1; TMPRSS4; ANGPT2; T1MP1; LOXL4; p53; IL-6; EGFR; Ku70; GST-pi; and polybromo-1D.

Biomarkers associated with development of colorectal cancers are shown in Raitano et al. (U.S. Pat. No. 7,507,541), Reinhard et al. (U.S. Pat. No. 7,501,244), Waldman et al. (U.S. Pat. No. 7,479,376); Schleyer et al. (U.S. Pat. No. 7,198,899); Reed (U.S. Pat. No. 7,163,801), Robbins et al. (U.S. Pat. No. 7,022,472), Mack et al. (U.S. Pat. No. 6,682,890), Tabiti et al. (U.S. Pat. No. 5,888,746), Budiman et al. (US 2009/0098542), Karl (US 2009/0075311), Arjol et al. (US 2008/0286801), Lee et al. (US 2008/0206756), Mori et al. (US 2008/0081333), Wang et al. (US 2008/0058432), Belacel et al. (US 2008/0050723), Stedronsky et al. (US 2008/0020940), An et al. (US 2006/0234254), Eveleigh et al. (US 2004/0146921), and Yeatman et al. (US 2006/0195269). The contents of each of the articles, patents, and patent applications are incorporated by reference herein in their entirety. Exemplary biomarkers that have been associated with colorectal cancers include: 36P6D5; TTK; CDX2; NRG4; TUCAN; hMLH1; hMSH2; M2-PK; CGA7; CJA8; PTP.alpha.; APC; p53; Ki-ras; complement C3a des-arg; alpha1-antitrypsin; transferrin; MMP-11; CA-19-9; TPA; TPS; TIMP-1; C10orf3; carcinoembryonic antigen (CEA); a soluble fragment of cytokeratin 19 (CYFRA 21-1); TAC1; carbohydrate antigen 724 (CA72-4); nicotinamide N-methyltransferase (NNMT); pyrroline-5-carboxylate reductase (PROC); S-adenosylhomocysteine hydrolase (SAHH); IBABP-L polypeptide; and Septin 9.

Biomarkers associated with development of prostate cancer are shown in Sidransky (U.S. Pat. No. 7,524,633), Platica (U.S. Pat. No. 7,510,707), Salceda et al. (U.S. Pat. No. 7,432,064 and U.S. Pat. No. 7,364,862), Siegler et al. (U.S. Pat. No. 7,361,474), Wang (U.S. Pat. No. 7,348,142), Ali et al. (U.S. Pat. No. 7,326,529), Price et al. (U.S. Pat. No. 7,229,770), O'Brien et al. (U.S. Pat. No. 7,291,462), Golub et al. (U.S. Pat. No. 6,949,342), Ogden et al. (U.S. Pat. No. 6,841,350), An et al. (U.S. Pat. No. 6,171,796), Bergan et al. (US 2009/0124569), Bhowmick (US 2009/0017463), Srivastava et al. (US 2008/0269157), Chinnaiyan et al. (US 2008/0222741), Thaxton et al. (US 2008/0181850), Dahary et al. (US 2008/0014590), Diamandis et al. (US 2006/0269971), Rubin et al. (US 2006/0234259), Einstein et al. (US 2006/0115821), Paris et al. (US 2006/0110759), Condon-Cardo (US 2004/0053247), and Ritchie et al. (US 2009/0127454). The contents of each of the articles, patents, and patent applications are incorporated by reference herein in their entirety. Exemplary biomarkers that have been associated with prostate cancer include: PSA; GSTP1; PAR; CSG; MIF; TADG-15; p53; YKL-40; ZEB; HOXC6; Pax 2; prostate-specific transglutaminase; cytokeratin 15; MEK4; MIP1-β; fractalkine; IL-15; ERGS; EZH2; EPC1; EPC2; NLGN-4Y; kallikrein 11; ABP280 (FLNA); AMACR; AR; BM28; BUB3; CaMKK; CASPASE3; CDK7; DYNAMIN; E2F1; E-CADHERIN; EXPORTIN; EZH2; FAS; GAS7; GS28; ICBP90; ITGA5; JAGGED1; JAM1; KANADAPTIN; KLF6; KRIP1; LAP2; MCAM; MIB1 (MKI67); MTA1; MUC1; MYOSIN-VI; P27; P63; P27; PAXILLIN; PLCLN; PSA(KLK3); RAB27; RBBP; RIN1; SAPKα; TPD52; XIAP; ZAG; and semenogelin II.

Biomarkers associated with development of pancreatic cancer are shown in Sahin et al. (U.S. Pat. No. 7,527,933), Rataino et al. (U.S. Pat. No. 7,507,541), Schleyer et al. (U.S. Pat. No. 7,476,506), Domon et al. (U.S. Pat. No. 7,473,531), McCaffey et al. (U.S. Pat. No. 7,358,231), Price et al. (U.S. Pat. No. 7,229,770), Chan et al. (US 2005/0095611), Mitchl et al. (US 2006/0258841), and Faca et al. (PLoS Med 5(6):e123, 2008). The contents of each of the articles, patents, and patent applications are incorporated by reference herein in their entirety. Exemplary biomarkers that have been associated with pancreatic cancer include: CA19.9; 36P6D5; NRG4; ASCT2; CCR7; 3C4-Ag; KLK11; Fibrinogen γ; and YKL40.

Biomarkers associated with development of lung cancer are shown in Sahin et al. (U.S. Pat. No. 7,527,933), Hutteman (U.S. Pat. No. 7,473,530), Bae et al. (U.S. Pat. No. 7,368,255), Wang (U.S. Pat. No. 7,348,142), Nacht et al. (U.S. Pat. No. 7,332,590), Gure et al. (U.S. Pat. No. 7,314,721), Patel (U.S. Pat. No. 7,300,765), Price et al. (U.S. Pat. No. 7,229,770), O'Brien et al. (U.S. Pat. No. 7,291,462 and U.S. Pat. No. 6,316,213), Muramatsu et al. (U.S. Pat. No. 7,090,983), Carson et al. (U.S. Pat. No. 6,576,420), Giordano (U.S. Pat. No. 5,840,506), Guo (US 2009/0062144), Tsao et al. (US 2008/0176236), Nakamura et al. (US 2008/0050378), Raponi et al. (US 2006/0252057), Yip et al. (US 2006/0223127), Pollock et al. (US 2006/0046257), Moon et al. (US 2003/0224509), and Budiman et al. (US 2009/0098543). The contents of each of the articles, patents, and patent applications are incorporated by reference herein in their entirety. Exemplary biomarkers that have been associated with lung cancer include: COX-2; COX4-2; RUNX3; aldoketoreductase family 1, member B 10; peroxiredoxin 1 (PRDX1); TNF receptor superfamily member 18; small proline-rich protein 3 (SPRR3); SOX1; SC6; TADG-15; YKL40; midkine; DAP-kinase; HOXA9; SCCE; STX1A; HIF1A; CCT3; HLA-DPB1; MAFK; RNF5; KIF11; GHSR1b; NTSR1; FOXM1; and PUMP-1.

Biomarkers associated with development of skin cancer (e.g., basal cell carcinoma, squamous cell carcinoma, and melanoma) are shown in Roberts et al. (U.S. Pat. No. 6,316,208), Polsky (U.S. Pat. No. 7,442,507), Price et al. (U.S. Pat. No. 7,229,770), Genetta (U.S. Pat. No. 7,078,180), Carson et al. (U.S. Pat. No. 6,576,420), Moses et al. (US 2008/0286811), Moses et al. (US 2008/0268473), Dooley et al. (US 2003/0232356), Chang et al. (US 2008/0274908), Alani et al. (US 2008/0118462), Wang (US 2007/0154889), and Zetter et al. (US 2008/0064047). The contents of each of the articles, patents, and patent applications are incorporated by reference herein in their entirety. Exemplary biomarkers that have been associated with skin cancer include: p27; Cyr61; ADAMTS-7; Cystatin B; Chaperonin 10; Profilin; BRAF; YKL-40; DDX48; erbB3-binding protein; biliverdin reductase; PLAB; LlCAM; SAA; CRP; SOX9; MMP2; CD10; and ZEB.

Biomarkers associated with development of multiple myeloma are shown in Coignet (U.S. Pat. No. 7,449,303), Shaughnessy et al. (U.S. Pat. No. 7,308,364), Seshi (U.S. Pat. No. 7,049,072), and Shaughnessy et al. (US 2008/0293578, US 2008/0234139, and US 2008/0234138). The contents of each of the articles, patents, and patent applications are incorporated by reference herein in their entirety. Exemplary biomarkers that have been associated with multiple myeloma include: JAG2; CCND1; MAF; MAFB; MMSET; CST6; RAB7L1; MAP4K3; HRASLS2; TRAIL; IG; FGL2; GNG11; MCM2; FLJ10709; TRIM13; NADSYN1; TRIM22; AGRN; CENTD2; SESN1; TM7SF2; NICKAP1; COPG; STAT3; ALOX5; APP; ABCB9; GAA; CEP55; BRCA1; ANLN; PYGL; CCNE2; ASPM; SUV39H2; CDC25A; IFIT5; ANKRA2; PHLDB1; TUBA1A; CDCA7; CDCA2; HFE; RIF1; NEIL3; SLC4A7; FXYD5; MCC; MKNK2; KLHL24; DLC1; OPN3; B3GALNT1; SPRED1; ARHGAP25; RTN2; WNT16; DEPDC1; STT3B; ECHDC2; ENPP4; SAT2; SLAMF7; MAN1C1; INTS7; ZNF600; L3 MBTL4; LAPTM4B; OSBPL10; KCNS3; THEX1. CYB5D2; UNC93B1; SIDT1; TMEM57; HIGD24; FKSG44; C14orf28; LOC387763; TncRNA; C18orf1; DCUN1D4; FANCI; ZMAT3; NOTCH1; BTG2; RAB1A; TNFRSF10B; HDLBP; RIT1; KIF2C; S100A4; MEIS1; SGOL2; CD302; COX2; C5orf34; FAM111B; C18orf54; and TP53.

Biomarkers associated with development of leukemia are shown in Ando et al. (U.S. Pat. No. 7,479,371), Coignet (U.S. Pat. No. 7,479,370 and U.S. Pat. No. 7,449,303), Davi et al. (U.S. Pat. No. 7,416,851), Chiorazzi (U.S. Pat. No. 7,316,906), Seshi (U.S. Pat. No. 7,049,072), Van Baren et al. (U.S. Pat. No. 6,130,052), Taniguchi (U.S. Pat. No. 5,643,729), Insel et al. (US 2009/0131353), and Van Bockstaele et al. (Blood Rev. 23(1):25-47, 2009). The contents of each of the articles, patents, and patent applications are incorporated by reference herein in their entirety. Exemplary biomarkers that have been associated with leukemia include: SCGF; JAG2; LPL; ADAM29; PDE; Cryptochrome-1; CD49d; ZAP-70; PRAME; WT1; CD15; CD33; and CD38.

Biomarkers associated with development of lymphoma are shown in Ando et al. (U.S. Pat. No. 7,479,371), Levy et al. (U.S. Pat. No. 7,332,280), and Arnold (U.S. Pat. No. 5,858,655). The contents of each of the articles, patents, and patent applications are incorporated by reference herein in their entirety. Exemplary biomarkers that have been associated with lymphoma include: SCGF; LMO2; BCL6; FN1; CCND2; SCYA3; BCL2; CD79a; CD7; CD25; CD45RO; CD45RA; and PRAD1 cyclin.

Biomarkers associated with development of bladder cancer are shown in Price et al. (U.S. Pat. No. 7,229,770), Orntoft (U.S. Pat. No. 6,936,417), Haak-Frendscho et al. (U.S. Pat. No. 6,008,003), Feinstein et al. (U.S. Pat. No. 6,998,232), Elting et al. (US 2008/0311604), and Wewer et al. (2009/0029372). The contents of each of the patent applications and each of these patents are incorporated by reference herein in their entirety. Exemplary biomarkers that have been associated with bladder cancer include: FGFR3, NT-3; NGF; GDNF; YKL-40; p53; pRB; p21; p27; cyclin E1; Ki67; Fas; urothelial carcinoma-associated 1; human chorionic gonadotropin beta type II; insulin-like growth factor-binding protein 7; sorting nexin 16; chondroitin sulfate proteoglycan 6; cathepsin D; chromodomain helicase DNA-binding protein 2; nell-like 2; tumor necrosis factor receptor superfamily member 7; cytokeratin 18 (CK18); ADAMS; ADAM10; ADAM12; Matrix Metalloproteinase-2 (MMP-2); MMP-9; KAI1; and bladder tumor fibronectin (BTF).

In certain circumstances, nucleic acids and proteins associated with a certain cancer vary with respect to the genetic, biochemical, or molecular alterations that associate the nucleic acid or protein with cancer. For example, the cancer causing alterations can include abnormal protein expressions, sequence mutations, methylation patterns, and loss of heterozygosity. Because multiple alterations can be linked to cancer, methods of the invention realize that there is great clinical value in assaying for multiple genetic characteristics across the plurality of biomarkers. In certain aspects, the invention involves obtaining a urine or tissue sample, conducting an assay on the urine or tissue sample to look for a nucleic acid mutation, loss of heterozygosity, and an abnormal protein level, and determining whether the sample is positive or negative for cancer based on the assay. By detecting different alterations in a signal assay, the result is a multimodal analysis that has greater sensitivity and specificity with regard to the diagnosis and characterization of the disease.

Methods of the invention provide for conducting an assay on a plurality of biomarkers to look for characteristics such as a nucleic acid mutation, a loss of heterozygosity, an abnormal protein level, gene expression patterns, an abnormal methylation pattern, and any other characteristic indicative of cancer. The presence or absence of one or more characteristic is indicative of a positive result for the cancer to be diagnosed. In certain embodiments, the type of characteristic looked for in the plurality of biomarkers is based on the cancer being diagnosed. For example, characteristics associated with bladder cancer include nucleic acid mutations, loss of heterozygosity, abnormal protein levels, and hypermethylation, whereas other cancer types might only be associated with abnormal protein level and hypermethylation patterns. Below the type of characteristics in proteins and nucleic acids that are suitable for use in methods of the invention are exemplified.

Nucleic acid biomarkers are often associated with nucleic acid mutations, which include additions, deletions, insertions, rearrangements, inversions, transitions, transversions, frameshift mutations, nonsense mutations, missense mutations, single nucleotide polymorphisms (SNP) and substitutions of two or more nucleotides within a sequence but not to the extent of large chromosomal sequence changes. SNPs are a type of genomic subtle sequence change that occurs when a single nucleotide replaces another within the sequence. Alterations in chromosome numbers include additions, deletions, inversions, translocations, copy number variations, and substitutions of chromosomes within a sequence. These nucleic acid mutations in biomarkers are often linked to cancer. For example, mutations of the FGFR3 gene and the p53 gene have been observed in bladder cancer. Cappellen D, De Oliveira C, Ricol D, et al., “Frequent activating mutations of FGFR3 in human bladder and cervix carcinomas.” NatGenet. 1999; 23(1):18-20; Berggren et al., “p53 mutations in urinary bladder cancer” British Journal of Cancer (2001) 84, 1505-1511. doi:10.1054/bjoc.2001.1823.

Loss of heterozygosity (LOH) is a common occurrence in patients with cancer. LOH indicates the absence of a functional tumor suppressor gene in the lost region. Loss of heterozygosity results from a deletion or other mutational event within a normal allele at a particular locus heterozygous for a deleterious mutant allele and the normal allele. The mutation in the normal allele renders the cell either hemizygous (one deleterious allele and one deleted allele) or homozygous for the deleterious allele. In other words, the loss of the normal allele is the LOH and may be a genetic determinant in the development of cancer. For example, loss of heterozygosity in the p53 gene is associated with bladder cancer. See Oka et al., “Detection of loss of heterozygosity in the p53 gene in renal cell carcinoma and bladder cancer using the polymerase chain reaction.” Molecular Carcinogenesis: Volume 4, Issue 1, 2006.

In certain embodiments, the level of protein biomarkers in the sample is analyzed in the multi-analyte screening assay to determine if there is an abnormal protein level in the sample. Protein biomarkers are generally considered quantitative biomarkers for which a level or amount of the biomarker present in comparison to a reference level or amount indicates a clinical status. For example, matrix metalloproteinases, such as MMP-2, MMP-9, and metalloproteases, such as ADAM-12, are associated with bladder cancer. MMPs have been shown to be key regulators of tumor growth, angiogenesis and metastasis formation. Increased MMP expression is required for tumors to grown into the surrounding tissue and for dissemination of metastatic cells into the vasculature and distant sites. Detection of MMPs in the urine of cancer patients has been shown to correlate with disease status in a variety of cancers, including bladder cancer. Biologically active MMP-2 and MMP-9 are found at higher levels and at greater frequency in urine of cancer patients than in healthy controls. In addition, ADAM12 is expressed in higher levels in cancer subjects than in healthy controls and is described in commonly-owned U.S. application Ser. No. 12/120,544.

In a particular embodiment, methods of the invention optionally include screening for the presence or absence of a methylation pattern in nucleic acid biomarkers, which includes screening nucleic acids for de-methylation, methylation, hypomethylation and hypermethylation. DNA methylation is an important regulator of gene transcription and a large body of evidence has demonstrated that aberrant DNA methylation is associated with unscheduled gene silencing, and the genes with high levels of 5-methylcytosine in their promoter region are transcriptionally silent. Aberrant DNA methylation patterns have been associated with a large number of human malignancies and found in two distinct forms: hypermethylation and hypomethylation compared to normal tissue. Hypermethylation is one of the major epigenetic modifications that repress transcription via promoter region of tumor suppressor genes. Hypermethylation typically occurs at CpG islands in the promoter region and is associated with gene inactivation. Global hypomethylation has also been shown to be causally related to the development and progression of cancer through different mechanisms. For example, a hypermethylation pattern of TWIST1, NID2, and vimentin detected in urine samples is indicative of a positive result for bladder cancer. See Renard I et al., Eur Urol. 2010; 58(1):96-104.

In another embodiment, the multi-analyte screening assay includes screening for gene expression of nucleic acids. Nucleic acid biomarkers associated with gene expression are generally considered quantitative biomarkers for which a level or amount of the biomarker present in comparison to a reference level or amount indicates a clinical status. For example, genes that exhibited significant over-expression in bladder cancer v.s. normal tissue include VEGFA, p16^{INK4A}, p53, EGFR, EGF, Ki-67, KRAS, NRAS, and cyclin D1. See, e.g. Zaravinos et al. “Spotlight on Differentially Expressed Genes in Urinary Bladder Cancer.” Cancer Epidemiol Biomarkers Prev. 2009 February; 18(2):444-53. Epub 2009 Feb. 3. The differential expression of these genes may be indicative of a positive result for cancer.

Nucleic acid biomarkers generally produce a binary result, i.e., presence or absence of an alteration or characteristic in the sample as compared to a healthy control is indicative of a clinical status. Protein biomarkers are generally considered quantitative biomarkers for which a level or amount of the biomarker present in comparison to a reference level or amount indicates a clinical status. As already discussed herein, threshold values for any particular biomarker and associated disease may be determined by reference to literature or standard of care criteria or may be determined empirically.

The following describes in detail the various types of assays suitable for use in methods of the invention.

Protein and nucleic acid biomarkers may be assayed or detected by any method known in the art for use in a single multi-analyte screening assay. Methods of the invention provide for conducting at least one detection assay on the plurality of biomarkers to look for any one of the characteristics indicative of cancer described above. Any combination of biomarkers or characteristics can be assayed using the same sequencing platform or different sequencing platforms. Accordingly, more than one detection technique can be conducted on the plurality of biomarkers to look for any variety of characteristics for the single multi-analyte screening assay. For example, one detection technique can be chosen because it is particularly suitable for detection of a particular biomarker and another detection technique can be chosen because it is particular suitable for detecting a particular characteristic.

In one embodiment, nucleic acids biomarkers are assayed using sequencing techniques and protein nucleic acid biomarkers are assayed using an array-based technique. For example, characteristics, such as nucleic acid mutations, methylation patterns and loss of heterozygosity, in nucleic acid biomarkers may be detected by using labeled probes or by sequencing, whereas abnormal protein levels can be detected in protein biomarkers using an array-based technique.

Methods of the invention also provide for conducting an assay in a tissue or a body fluid in order to determine an amount of two or more nucleic acids and one or more proteins in a sample using a single analytical platform, such as a qPCR assay or a single molecule sequencing technique. In such embodiment, protein levels of protein biomarkers are quantified on the same platform as nucleic acids by detecting aptamers that specifically bind to the protein to be detected. In another aspect of the invention, the assay on the protein biomarkers and nucleic acid biomarkers is conducted simultaneously, for example, by performing multiplex sequencing on a single analyte platform to determine a level of two or more nucleic acids and to determine a level of one or more proteins (via aptamer-based detection).

In one aspect of the invention, a single analytical assay is used to detect both nucleic acids and proteins from a single sample. Biological samples usually do not include a sufficient amount of DNA for detection. A common technique used to increase the amount of nucleic acid in a sample is to perform PCR on the sample prior to performing an assay that detects the nucleic acids in the sample. PCR involves thermal cycling, consisting of cycles of repeated heating and cooling of a reaction for DNA melting and enzymatic replication of the DNA. Most PCR protocols involve heating DNA to denature the double stranded DNA in the sample, cooling the DNA to allow for annealing of primers to the single-stranded DNA to form DNA/primer complexes and binding of a DNA polymerase to the DNA/primer complexes, and re-heating the sample so that the DNA polymerase synthesizes a new DNA strand complementary to the single-stranded DNA. This process amplifies the DNA in the sample and produces an amount of DNA sufficient for detection by standard assays known in the art, such as Southern blots or sequencing.

A problem with detecting both nucleic acids and proteins in a single assay is that the temperatures used for PCR adversely affect proteins in the sample, making the proteins undetectable by methods known in the art, such as western blots. For example, the required heating step in a PCR reaction brings the sample to a temperature that can result in irreversible denaturation of proteins in the sample and/or precipitation of proteins from the sample. Additionally, thermal cycling, i.e., repeated heating and cooling, can cause proteins in a sample to adopt a non-native tertiary structure. Once denatured, the proteins usually cannot be detected by standard protein assays such as western blots, immunoprecipitation, or immunoelectrophoresis. Therefore, a need exists for a single assay that can analyze both proteins and nucleic acids in a sample.

Methods of the present invention can detect a target nucleic acid and a target protein in a single assay. In certain embodiments, methods of the invention are accomplished by adding an aptamer to a sample that binds a target protein in the sample to form an aptamer/protein complex. An aptamer (nucleic acid ligand) is a nucleic acid macromolecule (e.g. DNA or RNA) that binds tightly to a specific molecular target, such as a protein. Since an aptamer is composed of DNA or RNA, it can be PCR amplified and can be detected by standard nucleic acid assays. PCR may then be used to amplify the nucleic acids and the aptamer in the sample. The amplified nucleic acids and aptamer may then be detected using standard techniques for detecting nucleic acids that are known in the art. In particular embodiments, the detection method is sequencing. Detection of the aptamer in the sample indicates the presence of the target protein in the sample.

As used herein, “aptamer” and “nucleic acid ligand” are used interchangeably to refer to a nucleic acid that has a specific binding affinity for a target molecule, such as a protein. Like all nucleic acids, a particular nucleic acid ligand may be described by a linear sequence of nucleotides (A, U, T, C and G), typically 15-40 nucleotides long. Nucleic acid ligands can be engineered to encode for the complementary sequence of a target protein known to associate with the presence or absence of a specific disease.

In solution, the chain of nucleotides form intramolecular interactions that fold the molecule into a complex three-dimensional shape. The shape of the nucleic acid ligand allows it to bind tightly against the surface of its target molecule. In addition to exhibiting remarkable specificity, nucleic acid ligands generally bind their targets with very high affinity, e.g., the majority of anti-protein nucleic acid ligands have equilibrium dissociation constants in the picomolar to low nanomolar range.

Aptamers used in the methods of the invention depend upon the target protein to be detected. Nucleic acid ligands for specific target proteins may be discovered by any method known in the art. In one embodiment, nucleic acid ligands are discovered using an in vitro selection process referred to as SELEX (Systematic Evolution of Ligands by Exponential enrichment). See for example Gold et al. (U.S. Pat. Nos. 5,270,163 and 5,475,096), the contents of each of which are herein incorporated by reference in their entirety. SELEX is an iterative process used to identify a nucleic acid ligand to a chosen molecular target from a large pool of nucleic acids. The process relies on standard molecular biological techniques, using multiple rounds of selection, partitioning, and amplification of nucleic acid ligands to resolve the nucleic acid ligands with the highest affinity for a target molecule. The SELEX method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. There have been numerous improvements to the basic SELEX method, any of which may be used to discover nucleic acid ligands for use in methods of the invention. In certain embodiments, the aptamers are designed to specifically bind to MMP-2 or MMP-9.

In methods of the invention, aptamers are introduced to the sample to bind the target protein. Certain of the aptamers bind the protein(s) of interest in the sample to form aptamer/protein complexes. The unbound aptamers are then separated and/or removed from sample using standard methods known in the art. See for example, Schneider et al., U.S. Patent Application Publication Number 2009/0042206, the content of which is incorporated by reference herein in its entirety.

Amplification refers to production of additional copies of a nucleic acid sequence. See for example, Dieffenbach and Dveksler, PCR Primer, a Laboratory Manual, Cold Spring Harbor Press, Plainview, N.Y. (1995), the contents of which is hereby incorporated by reference in its entirety. The amplification reaction may be any amplification reaction known in the art that amplifies nucleic acid molecules, such as polymerase chain reaction, nested polymerase chain reaction, polymerase chain reaction-single strand conformation polymorphism, ligase chain reaction, strand displacement amplification and restriction fragments length polymorphism.

In certain methods of the invention, the target nucleic acid and the nucleic acid ligand are PCR amplified. PCR refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. The process for amplifying the target nucleic acid sequence and nucleic acid ligand includes introducing an excess of oligonucleotide primers that bind the nucleic acid and the nucleic acid ligand, followed by a precise sequence of thermal cycling in the presence of a DNA polymerase. The primers are complementary to their respective strands of the target nucleic acid and nucleic acid ligand.

To effect amplification, the mixture of primers are annealed to their complementary sequences within the target nucleic acid and nucleic acid ligand. Following annealing, the primers are extended with a polymerase so as to form a new pair of complementary strands. The steps of denaturation, primer annealing and polymerase extension can be repeated many times (i.e., denaturation, annealing, and extension constitute one cycle; there can be numerous cycles) to obtain a high concentration of an amplified segment of a desired target and nucleic acid ligand. The length of the amplified segment of the desired target and nucleic acid ligand is determined by relative positions of the primers with respect to each other, and therefore, this length is a controllable parameter.

With PCR, it is possible to amplify a single copy of a specific target sequence in genomic DNA to a level that can be detected by several different methodologies (e.g., staining, hybridization with a labeled probe, incorporation of biotinylated primers followed by avidin-enzyme conjugate detection, incorporation of 32P-labeled deoxynucleotide triphosphates, such as dCTP or dATP, into the amplified segment).

In one embodiment of the invention, the target nucleic acid and nucleic acid ligand can be detected using detectably labeled probes. Nucleic acid probe design and methods of synthesizing oligonucleotide probes are known in the art. See, e.g., Sambrook et al., DNA microarray: A Molecular Cloning Manual, Cold Spring Harbor, N.Y., (2003) or Maniatis, et al., Molecular Cloning: A Laboratory Manual, Cold Spring Harbor, N.Y., (1982), the contents of each of which are herein incorporated by reference herein in their entirety. Sambrook et al., Molecular Cloning: A Laboratory Manual (2^{nd} Ed.), Vols. 1-3, Cold Spring Harbor Laboratory, (1989) or F. Ausubel et al., Current Protocols In Molecular Biology, Greene Publishing and Wiley-Interscience, New York (1987), the contents of each of which are herein incorporated by reference in their entirety. Suitable methods for synthesizing oligonucleotide probes are also described in Caruthers, Science, 230:281-285, (1985), the contents of which are incorporated by reference.

Probes suitable for use in the present invention include those formed from nucleic acids, such as RNA and/or DNA, nucleic acid analogs, locked nucleic acids, modified nucleic acids, and chimeric probes of a mixed class including a nucleic acid with another organic component such as peptide nucleic acids. Probes can be single stranded or double stranded. Exemplary nucleotide analogs include phosphate esters of deoxyadenosine, deoxycytidine, deoxyguanosine, deoxythymidine, adenosine, cytidine, guanosine, and uridine. Other examples of non-natural nucleotides include a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, and any other structural moiety that can act substantially like a nucleotide or base, for example, by exhibiting base-complementarity with one or more bases that occur in DNA or RNA.

The length of the nucleotide probe is not critical, as long as the probes are capable of hybridizing to the target nucleic acid and nucleic acid ligand. In fact, probes may be of any length. For example, probes may be as few as 5 nucleotides, or as much as 5000 nucleotides. Exemplary probes are 5-mers, 10-mers, 15-mers, 20-mers, 25-mers, 50-mers, 100-mers, 200-mers, 500-mers, 1000-mers, 3000-mers, or 5000-mers. Methods for determining an optimal probe length are known in the art. See, e.g., Shuber, U.S. Pat. No. 5,888,778, hereby incorporated by reference in its entirety.

Probes used for detection may include a detectable label, such as a radiolabel, fluorescent label, or enzymatic label. See for example Lancaster et al., U.S. Pat. No. 5,869,717, hereby incorporated by reference. In certain embodiments, the probe is fluorescently labeled. Fluorescently labeled nucleotides may be produced by various techniques, such as those described in Kambara et al., Bio/Technol., 6:816-21, (1988); Smith et al., Nucl. Acid Res., 13:2399-2412, (1985); and Smith et al., Nature, 321: 674-679, (1986), the contents of each of which are herein incorporated by reference in their entirety. The fluorescent dye may be linked to the deoxyribose by a linker arm that is easily cleaved by chemical or enzymatic means. There are numerous linkers and methods for attaching labels to nucleotides, as shown in Oligonucleotides and Analogues: A Practical Approach, IRL Press, Oxford, (1991); Zuckerman et al., Polynucleotides Res., 15: 5305-5321, (1987); Sharma et al., Polynucleotides Res., 19:3019, (1991); Giusti et al., PCR Methods and Applications, 2:223-227, (1993); Fung et al. (U.S. Pat. No. 4,757,141); Stabinsky (U.S. Pat. No. 4,739,044); Agrawal et al., Tetrahedron Letters, 31:1543-1546, (1990); Sproat et al., Polynucleotides Res., 15:4837, (1987); and Nelson et al., Polynucleotides Res., 17:7187-7194, (1989), the contents of each of which are herein incorporated by reference in their entirety. Extensive guidance exists in the literature for derivatizing fluorophore and quencher molecules for covalent attachment via common reactive groups that may be added to a nucleotide. Many linking moieties and methods for attaching fluorophore moieties to nucleotides also exist, as described in Oligonucleotides and Analogues, supra; Guisti et al., supra; Agrawal et al, supra; and Sproat et al., supra

The detectable label attached to the probe may be directly or indirectly detectable. In certain embodiments, the exact label may be selected based, at least in part, on the particular type of detection method used. Exemplary detection methods include radioactive detection, optical absorbance detection, e.g., UV-visible absorbance detection, optical emission detection, e.g., fluorescence; phosphorescence or chemiluminescence; Raman scattering. Preferred labels include optically-detectable labels, such as fluorescent labels. Examples of fluorescent labels include, but are not limited to, 4-acetamido-4′-isothiocyanatostilbene-2,2′ disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; alexa; fluorescien; conjugated multi-dyes; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron.™. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Atto dyes, Cy3; Cy5; Cy5.5; Cy7; IRD 700; IRD 800; La Jolta Blue; phthalo cyanine; and naphthalo cyanine. Labels other than fluorescent labels are contemplated by the invention, including other optically-detectable labels.

Detection of a bound probe may be measured using any of a variety of techniques dependent upon the label used, such as those known to one of skill in the art. Exemplary detection methods include radioactive detection, optical absorbance detection, e.g., UV-visible absorbance detection, optical emission detection, e.g., fluorescence or chemiluminescence. Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993)), such as described in Yershov et al., Proc. Natl. Acad. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc.

In certain embodiments, the target nucleic acid or nucleic acid ligand or both are quantified using methods known in the art. A preferred method for quantitation is quantitative polymerase chain reaction (QPCR). As used herein, “QPCR” refers to a PCR reaction performed in such a way and under such controlled conditions that the results of the assay are quantitative, that is, the assay is capable of quantifying the amount or concentration of a nucleic acid ligand present in the test sample.

QPCR is a technique based on the polymerase chain reaction, and is used to amplify and simultaneously quantify a targeted nucleic acid molecule. QPCR allows for both detection and quantification (as absolute number of copies or relative amount when normalized to DNA input or additional normalizing genes) of a specific sequence in a DNA sample. The procedure follows the general principle of PCR, with the additional feature that the amplified DNA is quantified as it accumulates in the reaction in real time after each amplification cycle. QPCR is described, for example, in Kurnit et al. (U.S. Pat. No. 6,033,854), Wang et al. (U.S. Pat. Nos. 5,567,583 and 5,348,853), Ma et al. (The Journal of American Science, 2(3), (2006)), Heid et al. (Genome Research 986-994, (1996)), Sambrook and Russell (Quantitative PCR, Cold Spring Harbor Protocols, (2006)), and Higuchi (U.S. Pat. Nos. 6,171,785 and 5,994,056). The contents of these are incorporated by reference herein in their entirety.

Two common methods of quantification are: (1) use of fluorescent dyes that intercalate with double-stranded DNA, and (2) modified DNA oligonucleotide probes that fluoresce when hybridized with a complementary DNA.

In the first method, a DNA-binding dye binds to all double-stranded (ds)DNA in PCR, resulting in fluorescence of the dye. An increase in DNA product during PCR therefore leads to an increase in fluorescence intensity and is measured at each cycle, thus allowing DNA concentrations to be quantified. The reaction is prepared similarly to a standard PCR reaction, with the addition of fluorescent (ds)DNA dye. The reaction is run in a thermocycler, and after each cycle, the levels of fluorescence are measured with a detector; the dye only fluoresces when bound to the (ds)DNA (i.e., the PCR product). With reference to a standard dilution, the (ds)DNA concentration in the PCR can be determined. Like other real-time PCR methods, the values obtained do not have absolute units associated with it. A comparison of a measured DNA/RNA sample to a standard dilution gives a fraction or ratio of the sample relative to the standard, allowing relative comparisons between different tissues or experimental conditions. To ensure accuracy in the quantification, it is important to normalize expression of a target gene to a stably expressed gene. This allows for correction of possible differences in nucleic acid quantity or quality across samples.

The second method uses sequence-specific RNA or DNA-based probes to quantify only the DNA containing the probe sequence; therefore, use of the reporter probe significantly increases specificity, and allows for quantification even in the presence of some non-specific DNA amplification. This allows for multiplexing, i.e., assaying for several genes in the same reaction by using specific probes with differently colored labels, provided that all genes are amplified with similar efficiency.

This method is commonly carried out with a DNA-based probe with a fluorescent reporter (e.g. 6-carboxyfluorescein) at one end and a quencher (e.g., 6-carboxy-tetramethylrhodamine) of fluorescence at the opposite end of the probe. The close proximity of the reporter to the quencher prevents detection of its fluorescence. Breakdown of the probe by the 5′ to 3′ exonuclease activity of a polymerase (e.g., Taq polymerase) breaks the reporter-quencher proximity and thus allows unquenched emission of fluorescence, which can be detected. An increase in the product targeted by the reporter probe at each PCR cycle results in a proportional increase in fluorescence due to breakdown of the probe and release of the reporter. The reaction is prepared similarly to a standard PCR reaction, and the reporter probe is added. As the reaction commences, during the annealing stage of the PCR, both probe and primers anneal to the DNA target. Polymerization of a new DNA strand is initiated from the primers, and once the polymerase reaches the probe, its 5′-3′-exonuclease degrades the probe, physically separating the fluorescent reporter from the quencher, resulting in an increase in fluorescence. Fluorescence is detected and measured in a real-time PCR thermocycler, and geometric increase of fluorescence corresponding to exponential increase of the product is used to determine the threshold cycle in each reaction.

In certain embodiments, the QPCR reaction uses fluorescent Taqman™ methodology and an instrument capable of measuring fluorescence in real time (e.g., ABI Prism 7700 Sequence Detector; see also PE Biosystems, Foster City, Calif.; see also Gelfand et al., (U.S. Pat. No. 5,210,015), the contents of which is hereby incorporated by reference in its entirety). The Taqman™ reaction uses a hybridization probe labeled with two different fluorescent dyes. One dye is a reporter dye (6-carboxyfluorescein), the other is a quenching dye (6-carboxy-tetramethylrhodamine). When the probe is intact, fluorescent energy transfer occurs and the reporter dye fluorescent emission is absorbed by the quenching dye. During the extension phase of the PCR cycle, the fluorescent hybridization probe is cleaved by the 5′-3′ nucleolytic activity of the DNA polymerase. On cleavage of the probe, the reporter dye emission is no longer transferred efficiently to the quenching dye, resulting in an increase of the reporter dye fluorescent emission spectra.

The nucleic acid ligand of the present invention is quantified by performing QPCR and determining, either directly or indirectly, the amount or concentration of nucleic acid ligand that had bound to its probe in the test sample. The amount or concentration of the bound probe in the test sample is generally directly proportional to the amount or concentration of the nucleic acid ligand quantified by using QPCR. See for example Schneider et al., U.S. Patent Application Publication Number 2009/0042206, Dodge et al., U.S. Pat. No. 6,927,024, Gold et al., U.S. Pat. Nos. 6,569,620, 6,716,580, and 7,629,151, Cheronis et al., U.S. Pat. No. 7,074,586, and Ahn et al., U.S. Pat. No. 7,642,056, the contents of each of which are herein incorporated by reference in their entirety.

Detecting the presence of the aptamer in the analyzed sample directly correlates to the presence of the target protein in that sample. In some embodiments of the invention, the amount of aptamer present in the sample correlates to the signal intensity following the conduction of the PCR-based methods. The signal intensity of PCR depends upon the number of PCR cycles performed and/or the starting concentration of the aptamer. Since the sequence of the target protein is known to generate the aptamer, detection of that specific aptamer correlates to the presence of the target protein. Similarly, detection of the amplified target nucleic acid indicates the presence of the target nucleic acid in the sample analyzed.

In one embodiment of the invention, during amplification of the aptamer or target nucleic acid using standard PCR methods, one method for detection and quantification of amplified aptamer or target nucleic acid results from the presence of a fluorogenic probe. In one embodiment of the invention, the probe, which is specific for the aptamer, has a 6-carboxyfluorescein (FAM) moiety covalently bound to the 5-′end and a 6-carboxytetramethylrhodamine (TAMRA) or other fluorescent-quenching dye (easily prepared using standard automated DNA synthesis) present on the 3′-end, along with a 3′-phosphate to prevent elongation. The probe is added with 5′-nuclease to the PCR assays, such that 5′-nuclease cleavage of the probe-aptamer duplex results in release of the 5′-bound FAM moiety from the oligonucleotide probe. As amplification continues and more aptamer is replicated by the PCR or RT-PCR enzymes, more FAM is released per cycle and so intensity of fluorescence signal per cycle increases. The relative increase in FAM emission is monitored during PCR or RT-PCR amplification using an analytical thermal cycler, or a combined thermal cycler/laser/detector/software system such as an ABI 7700 Sequence Detector (Applied Biosystems, Foster City, Calif.). The ABI instrument has the advantage of allowing analysis and display of quantification in less than 60 s upon termination of the amplification reactions. Both detection systems employ an internal control or standard wherein a second aptamer sequence utilizing the same primers for amplification but having a different sequence and thus different probe, is amplified, monitored and quantitated simultaneously as that for the desired target molecule. See for example, “A Novel Method for Real Time Quantitative RT-PCR,” Gibson, U. et. al., 1996, Genome Res. 6:995-1001; Piatak, M. et. al., 1993, BioTechniques 14:70-81; “Comparison of the BI 7700 System (TaqMan) and Competitive PCR for Quantification of IS6110 DNA in Sputum During Treatment of Tuberculosis,” Desjardin, L.e. et. al., 1998, J. Clin. Microbiol. 36(7):1964-1968), the contents of which are incorporated by reference, herein in their entirety.

In another method for detection and quantification of aptamer during amplification, the primers used for amplification contain molecular energy transfer (MET) moieties, specifically fluorescent resonance energy transfer (FRET) moieties, whereby the primers contain both a donor and an acceptor molecule. The FRET pair typically contains a fluorophore donor moiety such as 5-carboxyfluorescein (FAM) or 6-carboxy-4,5-dichloro-2,7-dimethoxyfluorescein (JOE), with an emission maximum of 525 or 546 nm, respectively, paired with an acceptor moiety such as N′N′N′N′-tetramethyl-6-carboxyrhodamine (TAMRA), 6-carboxy-X-rhodamine (ROX) or 6-carboxyrhodamine (R6G), all of which have excitation maximum of 514 nm. The primer may be a hairpin such that the 5′-end of the primer contains the FRET donor, and the 3′-end (based-paired to the 5′-end to form the stem region of the hairpin) contains the FRET acceptor, or quencher. The two moieties in the FRET pair are separated by approximately 15-25 nucleotides in length when the hairpin primer is linearized. While the primer is in the hairpin conformation, no fluorescence is detected. Thus, fluorescence by the donor is only detected when the primer is in a linearized conformation, i.e. when it is incorporated into a double-stranded amplification product. Such a method allows direct quantification of the amount of aptamer bound to target molecule in the sample mixture, and this quantity is then used to determine the amount of target molecule originally present in the sample. See for example, Nazarenko, I. A. et al., U.S. Pat. No. 5,866,336, the contents of which is incorporated by reference in its entirety.

In another embodiment of the invention, the QPCR reaction using TaqMan™ methodology selects a TaqMan™ probe based upon the sequence of the aptamer to be quantified and generally includes a 5′-end fluor, such as 6-carboxyfluorescein, for example, and a 3′-end quencher, such as, for example, a 6-carboxytetramethylfluorescein, to generate signal as the aptamer sequence is amplified using PCR. As the polymerase copies the aptamer sequence, the exonuclease activity frees the fluor from the probe, which is annealed downstream from the PCR primers, thereby generating signal. The signal increases as replicative product is produced. The amount of PCR product depends upon both the number of replicative cycles performed as well as the starting concentration of the aptamer. In another embodiment, the amount or concentration of an aptamer affinity complex (or aptamer covalent complex) is determined using an intercalating fluorescent dye during the replicative process. The intercalating dye, such as, for example, SYBR™ green, generates a large fluorescent signal in the presence of double-stranded DNA as compared to the fluorescent signal generated in the presence of single-stranded DNA. As the double-stranded DNA product is formed during PCR, the signal produced by the dye increases. The magnitude of the signal produced is dependent upon both the number of PCR cycles and the starting concentration of the aptamer.

Nucleic acids and proteins may be obtained by methods known in the art. Generally, nucleic acids can be extracted from a biological sample by a variety of techniques such as those described by Maniatis, et al., Molecular Cloning: A Laboratory Manual, Cold Spring Harbor, N.Y., pp. 280-281, (1982), the contents of which is incorporated by reference herein in its entirety. Generally, proteins can be extracted from a biological sample by a variety of techniques such as 2-D electrophoresis, isoelectric focusing, and SDS Slab Gel Electrophoresis. See for example O'Farrell, J. Biol. Chem., 250: 4007-4021 (1975), Sambrook, J. et al., Molecular Cloning: a Laboratory Manual, 2nd Edition, Cold Spring Harbor Laboratory Press, Cold Spring Harbor, N.Y. (1989), Anderson et al., U.S. Pat. No. 6,391,650, Shepard, U.S. Pat. No. 7,229,789, and Han et al., U.S. Pat. No. 7,488,579 the contents of each of which is hereby incorporated by reference in its entirety.

In other embodiments, antibodies with a unique oligonucleotide tag are added to the sample to bind a target protein and detection of the oligonucleotide tag results in detection of the protein. The target protein is exposed to an antibody that is coupled to an oligonucleotide tag of a known sequence. The antibody specifically binds the protein, and then PCR is used to amplify the oligonucleotide coupled to the antibody. The identity of the target protein is determined based upon the sequence of the oligonucleotide attached to the antibody and the presence of the oligonucleotide in the sample. In this embodiment of the invention, different antibodies specific for the target protein are used. Each antibody is coupled to a unique oligonucleotide tag of known sequence. Therefore, more than one target protein can be detected in a sample by identifying the unique oligonucleotide tag attached to the antibody. See for example Kahvejian, U.S. Patent Application Publication Number 2007/0020650, hereby incorporated by reference.

In other embodiments of the invention, antibodies with a unique nucleotide tag are added to the sample to bind the target nucleic acid. As described above, different antibodies specific for the target nucleic acid are used, therefore, more than one target nucleic acid can be detected in a sample by identifying the unique oligonucleotide tag attached. Detection of the nucleotide tag may be done by methods known in the art, such as PCR, QPCR, fluorescent labeling, radiolabeling, biotinylation, Sanger sequencing, sequencing by synthesis, or Single Molecule Real Time Sequencing methods. For description of single molecule sequencing methods see for example, Lapidus, U.S. Pat. No. 7,666,593, Quake et al., U.S. Pat. No. 7,501,245, and Lapidus et al., U.S. Pat. Nos. 7,169,560 and 7,491,498, the contents of each of which are herein incorporated by reference.

Antibodies for use in the present invention can be generated by methods well known in the art. See, for example, E. Harlow and D. Lane, Antibodies, a Laboratory Model, Cold Spring Harbor Laboratory Press, Cold Spring Harbor, N.Y., (1988), the contents of which are hereby incorporated by reference in their entirety. In addition, a wide variety of antibodies are available commercially.

The antibody can be obtained from a variety of sources, such as those known to one of skill in the art, including but not limited to polyclonal antibody, monoclonal antibody, monospecific antibody, recombinantly expressed antibody, humanized antibody, plantibodies, and the like; and can be obtained from a variety of animal species, including rabbit, mouse, goat, rat, human, horse, bovine, guinea pig, chicken, sheep, donkey, human, and the like. A wide variety of antibodies are commercially available and a custom-made antibody can be obtained from a number of contract labs. Detailed descriptions of antibodies, including relevant protocols, can be found in, among other places, Current Protocols in Immunology, Coligan et al., eds., John Wiley & Sons (1999, including updates through August 2003); The Electronic Notebook; Basic Methods in Antibody Production and Characterization, G. Howard and D. Bethel, eds., CRC Press (2000); J. Coding, Monoclonal Antibodies: Principles and Practice, 3d Ed., Academic Press (1996); E. Harlow and D. Lane, Using Antibodies, Cold Spring Harbor Lab Press (1999); P. Shepherd and C. Dean, Monoclonal Antibodies: A Practical Approach, Oxford University Press (2000); A. Johnstone and M. Turner, Immunochemistry 1 and 2, Oxford University Press (1997); C. Borrebaeck, Antibody Engineering, 2d ed., Oxford university Press (1995); A. Johnstone and R. Thorpe, Immunochemistry in Practice, Blackwell Science, Ltd. (1996); H. Zola, Monoclonal Antibodies: Preparation and Use of Monoclonal Antibodies and Engineered Antibody Derivatives (Basics: From Background to Bench), Springer Verlag (2000); and S. Hockfield et al., Selected Methods for Antibody and Nucleic Acid Probes, Cold Spring Harbor Lab Press (1993).

In certain embodiments, the target nucleic acid or nucleic acid ligand or both are detected using sequencing. In those embodiments, the aptamer/protein complex may be dissociated, releasing the aptamer for the sequencing reaction. Sequencing-by-synthesis is a common technique used in next generation procedures and works well with the instant invention. However, other sequencing methods can be used, including sequence-by-ligation, sequencing-by-hybridization, gel-based techniques and others. In general, sequencing involves hybridizing a primer to a template to form a template/primer duplex, contacting the duplex with a polymerase in the presence of a detectably-labeled nucleotides under conditions that permit the polymerase to add nucleotides to the primer in a template-dependent manner. Signal from the detectable label is then used to identify the incorporated base and the steps are sequentially repeated in order to determine the linear order of nucleotides in the template. Exemplary detectable labels include radiolabels, florescent labels, enzymatic labels, etc. In particular embodiments, the detectable label may be an optically detectable label, such as a fluorescent label. Exemplary fluorescent labels include cyanine, rhodamine, fluorescien, coumarin, BODIPY, alexa, or conjugated multi-dyes. Numerous techniques are known for detecting sequences and some are exemplified below. However, the exact means for detecting and compiling sequence data does not affect the function of the invention described herein.

In a preferred embodiment, the target nucleic acids, nucleic acid ligands, or both are detected using single molecule sequencing. Advantageously, methods of the invention have found that single molecule sequencing of DNA or protein biomarkers (via nucleic acid ligands) from urine samples show an increased sensitivity as compared to qPCR-based assays of biomarkers from urine samples. In fact, single molecule sequencing of DNA and protein biomarkers in urine has comparable sensitivity as qPCR sequencing of DNA and protein biomarkers from tissue samples, as highlighted in Example 3 below. Accordingly, assays of the invention that detect biomarkers in urine samples have similar performance and sensitivity of invasive tissue-based assays.

An example of a single molecule sequencing technique suitable for use in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H⁺), which signal detected and recorded in a sequencing instrument. The signal strength is proportional to the number of nucleotides incorporated. User guides describe in detail the Ion Torrent protocol(s) that are suitable for use in methods of the invention, such as Life Technologies' literature entitled “Ion Sequencing Kit for User Guide v. 2.0” for use with their sequencing platform the Personal Genome Machine™ (PCG).

In one embodiment, single molecule sequencing is used to maximize detection of FGFR3 mutations by conducting the biomarker assay on the Ion Torrent PGM platform (Life Technologies) ultra-deep sequencing platform. A primary PCR step is carried out using chimeric primers containing a sequence specific portion for amplifying the exons of interest (Exons 7, 10, and 15) along with adapter sequences required for sequencing analysis. Sequence specific primers suitable for use in smFGFR3 can be designed using any method known in the art. In certain embodiments, the primer can vary in lengths between 16 bp to 22 bp. The primary consideration is the Tm of the sequence specific portion. For example, primers with target specific Tm values ranging from ˜52° C. to ˜68° C. generated successful amplification products with chimeric oligonucleotides. Another consideration for primer design is the size of the amplicon because PCR products generated from total urine DNA have decreased yields at sizes larger than 300 bp. Accordingly, in certain embodiments, FGFR3 amplicons are designed to be ˜100 bp or smaller to accommodate read lengths on the sequencing platform. Although the above example is directed towards single molecule detection of FGFR3, methods of the invention also provide for single molecule detection of other nucleic acids, such as TWIST1, VIM, and NID2, and proteins such as MMP-2, MMP-9, and ADAM-12, through detection of protein-specific aptamers.

Another example of a DNA sequencing technique that can be used in the methods of the provided invention is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a sequencing technology that can be used in the methods of the provided invention is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using an electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

In certain embodiments, methods of the invention provide for detection of methylation patterns in nucleic acids. Methods include a number of bisulfite treatment sequencing methods in which genomic DNA is isolated and treated with bisulfite. Bisulfite DNA sequencing utilizes bisulfite-induced modification of genomic DNA under conditions whereby unmethylated cytosine is converted to uracil. The bisulfite-modified sequence is then amplified by PCR with two sets of strand-specific primers to yield a pair of fragments, one from each strand, in which all uracil and thymine residues are amplified as thymine and only 5-methylcytosine residues are amplified as cytosine. The PCR products can be sequenced or can be cloned and sequenced to provide methylation maps of single DNA molecules. See Frommer, M. et al., Proc. Natl. Acad. Sci. 89: 1827-1831 (1992). In certain aspects, after the nucleic acids are bisulfite modified, a barcode be ligated to the bisulfite modified targets and the methylated sample library can be pooled with other target nucleic acids and/or aptamers for multiplex sequencing.

Perhaps the most widely-used method of probing methylation patterns is methylation specific PCR (MSP) which uses two sets of primers for an amplification reaction. One primer set is complimentary to sequences whose Cs are converted to Us by bisulfite, and the other primer set is complimentary to non-converted Cs. Using these two separate primer sets, both the methylated and unmethylated DNA are amplified. Comparison of the amplification products gives insight as to the methylation in a given sequence. See Herman et al., “Methylation-specific PCR: A novel PCR assay for methylation status of CpG islands,” P.N.A.S., vol. 93, p. 9821-26 (1996), which is incorporated herein by reference in its entirety. This technique can detect methylation changes as small as ±0.1%. In addition to methylation of CpG islands, many of the sequences surrounding clinically relevant hypermethylated CpG islands can also be hypermethylated, and are potential biomarkers.

Beyond MSP, it is also possible to measure methylation levels by using hybridization probes that are specific for the products of bisulfate-converted nucleic acids using real-time PCR with primers that not complimentary to the CpG island regions of interest, or primers that hybridize to sequences adjacent to the CpG islands. Methods of using primers having abasic and or mismatch regions corresponding to CpG islands are disclosed in U.S. patent application Ser. No. 13/472,209 “Primers for Analyzing Methylated Sequences and Methods of Use Thereof,” filed May 15, 2012, and incorporated by reference herein in its entirety. Additionally, it is possible to determine an amount of methylation by amplifying and directly sequencing nucleic acids by using single molecule sequencing.

Sequences can be read that originate from a single molecule or that originate from amplifications from a single molecule. Millions of independent amplifications of single molecules can be performed in parallel either on a solid surface or in tiny compartments in water/oil emulsion: The DNA sample to be sequenced can be diluted and/or dispersed sufficiently to obtain one molecule in each compartment. This dilution can be followed by DNA amplification to generate copies of the original DNA sequences and creating “clusters” of molecules all having the same sequence. These clusters can then be sequenced. Many millions of reads can be generated in one run. Sequence can be generated starting at the 5′ end of a given strand of an amplified sequence and/or sequence can be generated from starting from the 5′ end of the complementary sequence. In a preferred embodiment, sequence from strands is generated, i.e. paired end reads (see for example, Harris, U.S. Pat. No. 7,767,400).

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide corresponds to a specific nucleotide species if they share base-complementarity with respect to at least one base.

Nucleotides for nucleic acid sequencing according to the invention preferably, include a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′ disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron.™. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Kornberg and Baker, W.H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent.™. DNA polymerase, Cariello et al., 1991, Polynucleotides Res, 19: 4193, New England Biolabs), 9.degree.Nm.™. DNA polymerase (New England Biolabs), Stoffel fragment, ThermoSequenase® (Amersham Pharmacia Biotech UK), Therminator.™. (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent.™. DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al, 1998, Proc. Natl. Acad. Sci. USA 95:14250).

Both mesophilic polymerases and thermophilic polymerases are contemplated. Thermophilic DNA polymerases include, but are not limited to, ThermoSequenase®, 9.degree.Nm.™., Therminator.™., Taq, Tne, Tma, Pfu, Tfl, Tth, Tli, Stoffel fragment, Vent.™. and Deep Vent.™. DNA polymerase, KOD DNA polymerase, Tgo, JDF-3, and mutants, variants and derivatives thereof. A highly-preferred form of any polymerase is a 3′ exonuclease-deficient mutant.

Reverse transcriptases useful in the invention include, but are not limited to, reverse transcriptases from HIV, HTLV-1, HTLV-II, FeLV, Hy, SIV, AMV, MMTV, MoMuLV and other retroviruses (see Levin, Cell 88:5-8 (1997); Verma, Biochim Biophys Acta. 473:1-38 (1977); Wu et al., CRC Crit. Rev Biochem. 3:289-347 (1975)).

In a preferred embodiment, nucleic acid template molecules are attached to a substrate (also referred to herein as a surface) and subjected to analysis by single molecule sequencing as described herein. Nucleic acid template molecules are attached to the surface such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

Substrates are preferably coated to allow optimum optical processing and nucleic acid attachment. Substrates for use in the invention can also be treated to reduce background. Exemplary coatings include epoxides, and derivatized epoxides (e.g., with a binding molecule, such as an oligonucleotide or streptavidin).

Various methods can be used to anchor or immobilize the nucleic acid molecule to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the template or the 5′ end of the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

Any detection method can be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Acad. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophor identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

Some embodiments of the invention use non-optical detection methods such as, for example, detection using nanopores (e.g., protein or solid state) through which molecules are individually passed so as to allow identification of the molecules by noting characteristics or changes in various properties or effects such as capacitance or blockage current flow (see, for example, Stoddart et al, Proc. Nat. Acad. Sci., 106:7702, 2009; Purnell and Schmidt, ACS Nano, 3:2533, 2009; Branton et al, Nature Biotechnology, 26:1146, 2008; Polonsky et al, U.S. Application 2008/0187915; Mitchell & Howorka, Angew. Chem. Int. Ed. 47:5565, 2008; Borsenberger et al, J. Am. Chem. Soc., 131, 7530, 2009); or other suitable non-optical detection methods.

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

In some embodiments, a plurality of nucleic acid molecules being sequenced is bound to a solid support. To immobilize the nucleic acid on a solid support, a capture sequence/universal priming site can be added at the 3′ and/or 5′ end of the template. The nucleic acids may be bound to the solid support by hybridizing the capture sequence to a complementary sequence covalently attached to the solid support. The capture sequence (also referred to as a universal capture sequence) is a nucleic acid sequence complimentary to a sequence attached to a solid support that may dually serve as a universal primer. In some embodiments, the capture sequence is polyNₙ, wherein N is U, A, T, G, or C, e.g., 20-70, 40-60, e.g., about 50. For example, the capture sequence could be polyT₄₀₋₅₀ or its complement. As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair.

In some embodiments, a barcode sequence is attached to the nucleic acid, the aptamer, or both. See for example, Steinman et al. (PCT internal application number PCT/US09/64001), the content of which is incorporated by reference herein in its entirety.

### Kits

In one embodiment the present invention relates to a kit comprising a detection reagent which binds to any nucleic acid sequence of ADAM12, GSTP1, FGFR3, MMP2, TWIST1, NID2, Vimentin, and/or p53, and/or polypeptides encoded thereby for the determination of bladder cancer.

One embodiment of the present invention relates to a kit for screening for, assessing the prognosis of an individual with bladder cancer, which comprises a reagent selected from the group consisting of: (a) a reagent for detecting mRNA of the ADAM12, GSTP1, FGFR3, MMP2, TWIST1, NID2, Vimentin, and/or p53 gene; (b) a reagent for detecting protein levels of ADAM12, GSTP1, FGFR3, MMP2, TWIST1, NID2, Vimentin, and/or p53; and (c) a reagent for detecting the biological activity of the ADAM12, GSTP1, FGFR3, MMP2, TWIST1, NID2, Vimentin, and/or p53.

In one embodiment, the present invention provides kits for detecting one or more of the following: a mutation in the FGFR3 gene, methylation status of TWIST1, methylation status of NID2, methylation status of Vimentin, protein levels of MMP2, a loss of heterozygozity in p53, and expression levels of ADAM12 protein. Further embodiments of kits may include additional biomarkers. In certain embodiments, the present invention provides kits for measuring the expression of the protein and/or RNA products of ADAM12, GSTP1, FGFR3, MMP2, TWIST1, NID2, Vimentin, and/or p53 in combination with at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, all or any combinational biomarkers mentioned herein.

Kits encompassed by the invention comprise materials and reagents required for measuring the expression of such protein and RNA products. In specific embodiments, the kits may further comprise one or more additional reagents employed in the various methods, such as: (1) reagents for stabilizing and/or purifying RNA from the sample (2) primers for generating test nucleic acids; (3) dNTPs and/or rNTPs (either premixed or separate), optionally with one or more uniquely labelled dNTPs and/or rNTPs (e.g., biotinylated or Cy3 or Cy5 tagged dNTPs); (4) post synthesis labelling reagents, such as chemically active derivatives of fluorescent dyes; (5) enzymes, such as reverse transcriptases, DNA polymerases, and the like; (6) various buffer mediums, e.g., reaction, hybridization and washing buffers; (7) labelled probe purification reagents and components, like spin columns, etc.; and (8) protein purification reagents; (9) signal generation and detection reagents, e.g., streptavidin-alkaline phosphatase conjugate, chemifluorescent or chemiluminescent substrate, and the like.

In particular embodiments, the kits comprise prelabeled quality controlled protein and or RNA isolated from a sample (e.g., blood or chondrocytes or synovial fluid) for use as a control. In some embodiments, the kits are RT-PCR or qRT-PCR kits. In other embodiments, the kits are nucleic acid arrays and protein arrays. Such kits according to the subject invention will at least comprise an array having associated protein or nucleic acid members of the invention and packaging means therefore. Alternatively, the protein or nucleic acid members of the invention may be pre-packaged onto an array.

In some embodiments, the kits are quantitative RT-PCR kits. In one embodiment, the quantitative RT-PCR kit includes the following: (a) primers used to amplify each of a combination of biomarkers of the invention; (b) buffers and enzymes including an reverse transcriptase; (c) one or more thermos table polymerases; and (d) Sybr® Green. In another embodiment, the kit of the invention also includes (a) a reference control RNA and (b) a spiked control RNA.

The invention provides kits that are useful for (a) diagnosing individuals as having bladder cancer and/or early stage bladder cancer. The invention also provides kits that are useful for determining the likelihood of bladder cancer in patients presented with hematuria. Additional embodiments of the invention include kits that are useful for monitoring the recurrence of bladder cancer. For example, in a particular embodiment of the invention a kit is comprised a forward and reverse primer wherein the forward and reverse primer are designed to quantitate expression of all of the species of mRNA corresponding to each of the biomarkers as identified in accordance with the invention useful in determining whether an individual has bladder cancer and/or early stage bladder cancer or not. In certain embodiments, at least one of the primers is designed to span an exon junction.

The invention provides kits that are useful for detecting, diagnosing, monitoring and prognosing bladder cancer based upon the detection of protein or RNA products of ADAM12, GSTP1, FGFR3, MMP2, TWIST1, NID2, Vimentin, and/or p53, possibly in combination with at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, all or any combination of the combinatorial biomarkers of the invention in a sample.

In certain embodiments, such kits do not include the materials and reagents for measuring the expression of a protein or RNA product of a biomarker of the invention that has been suggested by the prior art to be associated with bladder cancer. In other embodiments, such kits include the materials and reagents for measuring the expression of a protein or RNA product of a combinatorial biomarker of the invention that has been suggested by the prior art to be associated with bladder cancer and at least 1, at least 2, at least 3, at least 4, at least 5, at least 6, at least 7, at least 8, at least 9, at least 10, at least 15, at least 20, at least 25, at least 30, at least 35, at least 40, at least 45 or more genes other than the combinatorial biomarkers of the invention.

The invention provides kits useful for monitoring the efficacy of one or more therapies that a subject is undergoing based upon detecting a protein or RNA product of ADAM12, GSTP1, FGFR3, MMP2, TWIST1, NID2, Vimentin, and/or p53, possibly in combination with any number of up to at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, all or any combination of the combinatorial biomarkers of the invention in a sample. In certain embodiments, such kits do not include the materials and reagents for measuring the expression of a protein or RNA product of a biomarker of the invention that has been suggested by the prior art to be associated with bladder cancer. In other embodiments, such kits include the materials and reagents for measuring the expression of a protein or RNA product of ADAM12, GSTP1, FGFR3, MMP2, TWIST1, NID2, Vimentin, and/or p53, possibly in combination with a biomarker that has been suggested by the prior art to be associated with bladder cancer and any number of up to at least 1, at least 2, at least 3, at least 4, at least 5, at least 6, at least 7, at least 8, at least 9, at least 10, at least 15, at least 20, at least 25, at least 30, at least 35, at least 40, at least 45 or more genes other than the combinatorial biomarkers of the invention.

The invention provides kits useful for determining whether a subject will be responsive to a therapy based upon detecting a protein or RNA product of ADAM12, GSTP1, FGFR3, MMP2, TWIST1, NID2, Vimentin, and/or p53, possibly in combination with any number of up to at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, all or any combination of the combinatorial biomarkers of the invention in a sample.

In a specific embodiment, such kits comprise materials and reagents that are necessary for measuring the expression of a RNA product of a biomarker of the invention. For example, a kit may comprise a microarray or RT-PCR kit. For nucleic acid microarray kits, the kits generally comprise probes attached to a solid support surface. The probes may be labelled with a detectable label. In a specific embodiment, the probes are specific for an exon(s), an intron(s), an exon junction(s), or an exon-intron junction(s)), of RNA products of ADAM12 possibly in combination with any number of up to 1, 2, 3, 4, 5, 6, 7, 8, 9, 10, 15, 20, 25, 30, 35, 40, 45, 50, all or any combination of the combinatorial biomarkers of the invention.

The microarray kits may comprise instructions for performing the assay and methods for interpreting and analyzing the data resulting from the performance of the assay. In a specific embodiment, the kits comprise instructions for diagnosing bladder cancer. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

For RT-PCR kits, the kits generally comprise pre-selected primers specific for particular RNA products (e.g., an exon(s), an intron(s), an exon junction(s), and an exon-intron junction(s)) of ADAM12, GSTP1, FGFR3, MMP2, TWIST1, NID2, Vimentin, and/or p53 possibly in combination with any number of up to 1, 2, 3, 4, 5, 6, 7, 8, 9, 10, 15, 20, 25, 30, 35, 40, 45, 50, all or any combination of the combinatorial biomarkers of the invention. The RT-PCR kits may also comprise enzymes suitable for reverse transcribing and/or amplifying nucleic acids (e.g., polymerases such as Taq), and deoxynucleotides and buffers needed for the reaction mixture for reverse transcription and amplification. The RT-PCR kits may also comprise probes specific for RNA products of ADAM12, GSTP1, FGFR3, MMP2, TWIST1, NID2, VIMENTIN, and/or p53, and possibly any number of up to 1, 2, 3, 4, 5, 6, 7, 8, 9, 10, 15, 20, 25, 30, 35, 40, 45, 50, all or any combination of the combinatorial biomarkers of the invention. The probes may or may not be labelled with a detectable label (e.g., a fluorescent label). Each component of the RT-PCR kit is generally in its own suitable container. Thus, these kits generally comprise distinct containers suitable for each individual reagent, enzyme, primer and probe. Further, the RT-PCR kits may comprise instructions for performing the assay and methods for interpreting and analyzing the data resulting from the performance of the assay. In a specific embodiment, the kits contain instructions for diagnosing bladder cancer.

In a specific embodiment, the kit is a real-time RT-PCR kit. Such a kit may comprise a 96 well plate and reagents and materials necessary for e.g. SYBR Green detection. The kit may comprise reagents and materials so that beta-actin can be used to normalize the results. The kit may also comprise controls such as water, phosphate buffered saline, and phage MS2 RNA. Further, the kit may comprise instructions for performing the assay and methods for interpreting and analyzing the date resulting from the performance of the assay. In a specific embodiment, the instructions state that the level of a RNA product of ADAM12, GSTP1, FGFR3, MMP2, TWIST1, NID2, Vimentin, and/or p53, and possibly any number of up to 1, 2, 3, 4, 5, 6, 7, 8, 9, 10, 15, 20, 25, 30, 35, 40, 45, 50, all or any combination of the combinatorial biomarkers of the invention should be examined at two concentrations that differ by, e.g., 5 fold to 10-fold. For antibody based kits, the kit can comprise, for example: (1) a first antibody (which may or may not be attached to a solid support) which binds to ADAM12, GSTP1, FGFR3, MMP2, TWIST1, NID2, Vimentin, and/or p53 and any combinatorial protein of interest (e.g., a protein product of any number of up to 1, 2, 3, 4, 5, 6, 7, 8, 9, 10, 15, 20, 25, 30, 35, 40, 45, 50, all or any combination of the combinatorial biomarkers of the invention); and, optionally, (2) a second, different antibody which binds to either the protein, or the first antibody and is conjugated to a detectable label (e.g., a fluorescent label, radioactive isotope or enzyme). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay.

In a specific embodiment, the kits contain instructions for diagnosing bladder cancer.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### Example 1

### Detection of Bladder Cancer Recurrence

Urine samples were collected from 323 patients. All patients were previously treated for bladder cancer and were undergoing routine monitoring for recurrence. 48 of the patients were identified by various means to have a bladder cancer recurrence (all tumors confirmed by pathology) and 275 patients had no evidence of the disease at the given monitoring interval. Urine samples were aliquoted and stored at −80° C. until assayed. Urine samples for DNA analysis were stabilized with 25 mM EDTA prior to aliquoting and freezing. Primer sequences used in the method described below are provided in Appendix A.

Total MMP2 levels were determined by processing 50 mL neat urine through an MMP2 specific ELISA per the manufacturer's instructions (R&D Systems, Minneapolis, Minn.).

FGFR3 mutations were detected by utilizing a PCR-clamping methodology. Genomic DNA was first isolated from thawed urine samples using the QIAamp Minelute Virus Vacuum Kit per manufacturer's instructions. Only 316 of the 323 samples tested had sufficient DNA to reliably obtain a FGFR3 result. Primary PCR of genomic DNA extracted from 4 ml urine was carried out using oligonucleic primers specific for FGFR3 to amplify DNA from exons 7, 10, and 15. PCR amplification was performed using a C1000 thermal cycler (Bio-Rad laboratories, Hercules, Calif.) under standard conditions. DNA amplication was confirmed via agarose gel analysis of primary PCR products.

Wild-type nucleic acids containing locked nucleic acid (LNA) bases surrounding known mutation sites were included along with real-time PCR primers and dual-labeled Taqman probes. Real-time PCR amplification was performed using a Light Cycler real-time thermal cycler (Roche Diagnostics Corporation, Indianapolis, Ind.). Dual real-time PCR reactions, with and without the LNA blocker, were assembled in duplicate for each amplification.

Detection of methylated TWIST1 and NID2 was conducted using conventional methylation specific PCR (MSP). DNA was extracted from 8 mL urine as above and eluted in water. DNA yield was determined by quantitative real-time PCR using a reference gene. The DNA was then concentrated using AES 1000 Speed Vac (Thermo Fisher, Waltham Mass.) and resuspended in water. Bisulfite conversion of the DNA was performed using a Qiagen Epitect Bisulfite Kit per manufacturer's instructions. The converted DNA was subsequently loaded on columns, subjected to desulfonation, washed, and eluted in 30 ul molecular grade water and stored at −20° C. until assayed.

Conventional MSP was performed using methylation-specific primers to sequences within the promoter region of TWIST1 and NID2. PCR amplifications were performed using the C1000 thermocycler under standard conditions. Actin B quantitation was included as an assay control. Real-time PCR amplifications were conducted using a Roche LightCycler 480 under standard conditions.

Samples positive for FGFR3 mutation were assigned a score of “1,” while negative samples were assigned a score of “0.” For quantitative markers, i.e., MMP2, individual marker cutoffs were established to maximize specificity. Each marker was then scored as a “1” for above the cutoff or “0” for below the cutoff. The sum of all markers was used to establish a final clinical performance. To maximize NPV, samples were considered negative when a total score of “0” was obtained. Patients with scores of “0” could be excluded from further testing with very high NPV. Patients with scores greater than or equal to “1” were considered intermediate and should remain in cue for standard of care. Final clinical performance of sensitivity, specificity, and NPV were calculated using standard methods. Confidence intervals were calculated using an excel macro binomial confidence interval calculator.

As shown in Table 1, the combination of all four markers as described resulted in 97.4% NPV at a sensitivity of 92% with the possibility of excluding 51% of patients who do not have cancer from receiving further tests. Importantly, the three false negative samples observed here were all of low stage and grade (TaG1).

TABLE 1

| | | | | Power of |
|---|---|---|---|---|
| Biomarker | Cutoffs | NPV | Sensitivity | Exclusion |
| MMP2 | MMP2 < | 91.7% | 90% | 19% |
| | 0.309 ng/ml | | (43/48) | (51/268) |
| | | | [77%-97%] | [15%-24%] |
| MMP2 + NID2 | MMP2 < | 93.6% | 90% | 26% |
| | 0.418 ng/ml | | (43/48) | (71/268) |
| | NID2 < 600k | | [77%-97% | (21%-32%) |
| MMP2 + NID2 + | MMP2 < | 94.3% | 90% | 29% |
| FGFR3 | 0.456 ng/ml | | (43/48) | (77/268) |
| | NID2 < 600k | | [77%-97%] | [23%-35%] |
| MMP2 + NID2 + | MMP2 < | 97.4% | 92% | 51% |
| FGFR3 + | 1.100 ng/ml | | (44/48) | (136/268) |
| TWIST1 | NID2 < 600k | | [80%-98%] | [44%-57%] |
| | TWIST1 < | | | |
| | 249k | | | |

Based on these results, a noninvasive diagnostic test for the detection of cancer is provided. The particular methods described here are also useful in monitoring the recurrence of cancer. The presented assay combines the sensitivity of protein markers with the specificity of DNA markers for optimized clinical performance. Analysis of MMP2 protein levels is coupled with methylation analysis of TWIST1 and NID2 and mutational analysis of FGFR3. Using this approach, 51% of the patients being monitored for bladder cancer recurrence, but who do not have cancer, could have been excluded from further invasive intervention with very high confidence (97%). The described assay also allows patients to be stratified into three groups: one that is cancer-free and could be excluded from undergoing further evaluation; a second group that simply receives the already scheduled standard of care; and a third that has a high likelihood of cancer and could receive accelerated intervention.

The above example is exemplified in Fernandez et al., A noninvasive multi-analyte diagnostic assay: combining protein and DNA markers to stratify bladder cancer patients, Research and Reports in Urology 2012:4; 17-26, the entirety of which is incorporated by reference.

It is to be understood that the various DNA and protein biomarkers used in this example are not limiting and that the use of other biomarkers are contemplated with the described methods. It has been found, for example, that the combination of p53, FGFR3, MMP2, NID2, and Vimentin in a multi-analyte diagnostic assay for monitoring cancer recurrence provides particularly high sensitivity and NPV. Certain assays may incorporate the detection of MMP9 protein levels rather than MMP2. In addition, the described methods are not limited to bladder cancer, or even cancer in general, and can be used in other disease indications.

### Example 2

### Detection of Bladder Cancer in Patients Presented with Hematuria

Urine samples were obtained from 48 cancer patients and 256 patients who were evaluated for hematuria but who did not have cancer upon cytoscopic evaluation (Hem+/Cysto−). As described in Example 1, TWIST1 and NID2 methylation status was assessed using methylation-specific PCR primers, FGFR3 mutational status was determined by quantitative PCR, and MMP levels were determined by ELISA. Results are provided in Tables 2 and 3

TABLE 2

| | | | Power |
|---|---|---|---|
| Marker | Cutoff | Sensitivity | of Exclusion |
| TWIST1 | TWIST < 139k | (38/45) | 82% |
| | | [71-94%] | (201/2460 |
| | | | [76-86%] |
| NID2 | NID2 < 680k | 33% | 100% |
| | | (16/48) | (246/246) |
| | | [20-48%] | [99-100%] |
| FGFR3 | N/A | 10% | 99% |
| | | (5/48) | (244/246) |
| | | [3-23%] | [97-100%] |
| MMP2 | MMP2 < 1.100 | 35% | 74% |
| | | (17/48) | (181/246) |
| | | [22-51%] | [68-79%] |

TABLE 3

| | | NPV (adjusted to | | Power of |
|---|---|---|---|---|
| Markers | Cutoffs | 5% prevalence) | Sensitivity | Exclusion |
| TWIST1 + | TWIST < 139k | 99.5% | 94% | 65% |
| NID2 + | NID < 680k | | (45/48) | (159/246) |
| FGFR3 + | MMP2 < 1.100 | | [83-99%] | [58-71%] |
| MMP2 | | | | |

As shown in the Tables 2 and 3, the combined biomarker assay is able to provide a level of sensitivity (94%) not attainable with any one marker alone. The high DNA marker sensitivity allowed for higher MMP cutoffs to be set. The combined sensitivity of all four markers, although individually low, results in 94% sensitivity and the exclusion of 65% cancer-free patients from receiving further intervention with very high confidence (99.5% NPV). Accordingly, the methods disclosed in accordance with the present invention combines the better performance characteristics of protein and DNA biomarkers into one assay for optimized clinical performance. With the methods provided, the detection of FGFR3 mutations along with TWIST1 and NID2 methylation in the urine of hematuria patients effectively increases sensitivity and NPV at an established MMP cutoff. This noninvasive urinary diagnostic assay could be used to more efficiently triage hematuria patients by identifying those patients who do not have cancer and who could be excluded from receiving invasive procedures.

### Example 3

### Single Molecule Sequencing of FGFR3 Mutations

FGFR3 mutations have been identified in ˜60-70% of low-stage, non-invasive tumors. Conventional urine based assays for detecting FGFR3 mutations have been limited by the technical ability to detect rare events in a dilute medium where there is a high background of normal DNA. In these assays, FGFR3 mutations are generally found in ˜30% of the urine samples, which is <50% concordance with the expected detection in tissue. The following describes a method incorporating single molecule sequencing for improved detection of FGFR3 mutations.

Urine samples from 43 patients with bladder cancer were analyzed using the qPCR methods described in Example 1 and the single molecule sequencing approach described herein. For the single molecule sequencing analysis, amplicons were designed against FGFR3 exons 7, 10, and 15 using PCR primers containing the adapter sequences for unidirectional sequencing. Primary amplification was performed from DNA isolated from 4 ml urine. The resulting PCR products were used as templates for emulsion PCR and these were then sequenced using the Roche 454 GS Junior for the single molecule sequencing step. The Ion Torrent platform was also tested for the sequencing step.

Detection of mutations in the exon 7 region is shown in Table 4 below. Using the Roche 454 platform or the Ion Torrent platform, very low levels of mutant DNA were detectable in a predominantly normal background. These results indicate that the use of the single molecule sequencing methods described herein will increase analytical sensitivity.

TABLE 4

| | Exon | Mutant | Percent Mutant |
|---|---|---|---|
| | Specific Reads | Positive Reads | Detected |
| Roche 454 Platform | | | |
| Exon 7 | 34,489 | 6 | 0.02% |
| Exon 10 | 24,202 | 0 | 0.00% |
| Exon 15 | 9,975 | 0 | 0.00% |
| Ion Torrent Platform | | | |
| Exon 7 | 171,804 | 28 | 0.016% |
| Exon 10 | 161,911 | 0 | 0.00% |
| Exon 15 | 154,734 | 0 | 0.00% |

Nineteen matched tissue and urine samples were tested for FGFR3 mutations. As shown in Table 5 below, mutations were detected by qPCR in 11 of 19 tissue samples. However, mutations were only detected in 6 out of 19 urine samples using the same assay, suggesting a concordance of ˜50%. Using single molecule sequencing of FGFR3, mutations were detected in 15 out of 19 urine samples. 10 of those 15 were also detected in the tissue samples, resulting in 90% concordance.

TABLE 5

| | Tissue Using | Urine Using | Urine Using Single |
|---|---|---|---|
| | qPCR | qPCR | Molecule Sequencing |
| Sensitivity | 58% | 32% | 79% |
| | (11/19) | (6/19) | (15/19) |
| | [33-77%] | [15-54%] | [57-92%] |
| Concordance with | N/A | 46% | 91% |
| Tissue | | (5/11) | (10/11) |
| | | [21-72%] | [62-98%] |

As shown in Table 6 below, the increased analytical activity of the single molecule assay resulted in increased clinical sensitivity if FGFR3 mutations in urine. Accordingly, the methods described herein encompass a highly sensitive non-invasive assay in which mutations can be detected using single molecule sequencing. Furthermore, such methods can be incorporated into multi-analyte diagnostic assays.

TABLE 6

Sensitivity

| Cancer Stage | qPCR | Single Molecule Sequencing |
|---|---|---|
| Ta | 11.1% | 63.0% |
| | (3/27) | (17/27) |
| T1 | 22.2% | 55.6% |
| | (2/9) | (5/9) |
| ≧T2 | 0.0% | 28.6% |
| | (0/7) | (2/7) |
| All Stages | 11.6% | 55.8% |
| | (5/43) | (24/43) |
| | [5-24%] | [40-71%] |

### Example 4

### Enhancing Assay Performance with Single Molecule Sequencing and a Combined Single Molecule Assay for FGFR3 and p53

FGFR3 alone and in combination with p53 was assayed using qPCR and single molecule sequencing to determine if single molecule sequencing increased performance and enhanced sensitivity and to determine if a composite assay of p53 and FGFR3 has increased predictive performance over a single FGFR3 assay.

In previous qPCR assays, about 60% of FGFR3 mutations were consistently detected in bladder tumor tissue using qPCR, whereas only about 30% of FGFR3 mutations were found in urine using qPCR. It was hypothesized that the qPCR-based assays were not analytically sensitive to detect all the expected mutations in urine as the mutations found in tissue because of the very low mutant to normal DNA ratio found in urine in comparison to tissue. However, methods of the invention have found that performing a deep-sequencing assay, such as single molecule sequencing, to detect FGFR3 mutations in urine has enhanced detection performance over qPCR assays in tissue and urine.

For example, matched tissue and urine samples from 19 patients were used to determine urine/tissue concordance for the smFGFR3 assay in comparison to qPCR-based assays. In addition, 43 urine samples from the test set were used to determine the clinical performance of the smFGFR3 assay.

Table 7 shows the sensitivity of qPCR and smFGFR3 of DNA isolated from urine and a qPCR analysis of matched tissue sample.

TABLE 7

| | qPCR-tumor | qPCR-urine | smFGFR2-urine |
|---|---|---|---|
| Sensitivity | 58% (11/19) | 32% (6/9) | 79% (15/19) |
| Concordance | | 46% (5/11) | 91% (10/11) |

In Table 7, the detection of FGFR3 mutations in the 19 tissue samples is consistent with the 60% frequency of FGFR3 mutations found in previous qPCR assays. Of these, only 46% of mutations are detected in the matching urine samples by qPCR. In contrast, smFGFR3 assay detected mutations in the urine of 91% of the positive tumors. In addition, the smFGFR3 assay detected mutations in 5 samples that were negative for FGFR3 mutations in tissue. This possibility reflects sampling issues related to tumor heterogeneity or stochastic sampling, and also suggests that with such high analytical sensitivity, a noninvasive urine assay bay be more representative of the entire urothelium than analysis of the tumor or biopsy sections.

Table 8 shows the sensitivity of smFGFR3 in urine samples from 43 bladder cancer patients at different tumor stages.

TABLE 8

| Tumor | qPCR | smFGFR3 |
|---|---|---|
| Ta | 11.1% (3/27) | 63.0% (17/27) |
| T1 | 22.2% (2/9) | 55.6% (5/9) |
| ≧T2 | 0% (0/7) | 28.6% (2/7) |
| Total | 11.6% (5/43) | 55.8% (24/43) |

As shown in Table 8, the qPCR assay identified 5 samples as positive for FGFR3 mutations of the samples analyzed. The smFGFR3 identified these 5 patients and also an additional 19 patients as having FGFR3 mutation DNA that were present at <1% and as low as 0.02% of the total urine DNA. The clinical sensitivity of smFGFR3 was 55.8%, which is more representative of the frequency of FGFR3 mutations detected in tumor tissues. In contrast, the qPCR assay has lower analytical sensitivity and resulted in clinical sensitivity of only 11.6%. Out of the 27 Hematuria+/cystoscopy− samples tested, none were positive for mutations (100% specificity). These results demonstrate that superior analytical sensitivity will ultimately improve clinical performance.

Based on the performance of smFGFR3 in the urine samples depicted in Table 8, the expected performance of smFGFR3 for 58 cancer patients was calculated alone and in combination with small molecule detection of p53. The combination of FGFR3 and p53 was chosen because the genes are associated with two distinct pathways of bladder tumor development. Using smFGFR3 in combination p53 is projected to further increase the sensitivity of the FGFR3 bladder cancer assay without decreasing specificity. Table 9 shows the projected impact of smFGFR3 and smp53.

TABLE 9

| | | | smp53 + smFGFR3 |
|---|---|---|---|
| Stage | smFGFR3 (projected) | smp53 (projected) | (projected) |
| Ta | 61.1% (22/36) | 2.8% (1/36) | 69.4% (25/36) |
| T1 | 53.3% (8/15) | 20.0% (3/15) | 80.0% (12/15) |
| ≧T2 | 28.6% (2/7) | 28.9% (2/7) | 57.1% (4/7) |
| Total | 55.2% (32/58) | 10.3% (6/58) | 65.5% (38/58) |

As with smFGFR3 and given that p53 mutations are indicative of cancer, it is expected that p53 mutation detection in urine will increase sensitivity without decreasing specificity. At the expected frequency of p53 mutations in tissues that are negative for FGFR3 mutations, the combination of smFGFR3 and smp53 would identify in urine about 65% of all cancers.

In conclusion, the smFGFR3 assay of the invention identifies mutations with frequencies similar to those found in tissue-based assays and increases clinical sensitivity in urine-based assays. Detection of p53, although not high, complements FGFR3 detection in that there is little overlap between the mutations observed. The combination of smFGFR3 and p53 is expected to increase sensitivity without any loss of specificity.

In certain embodiments, the combined assay of smFGFR3 and smp53 is further combined with MMP-2, MMP-9, TWIST1, NID2, VIM and any combination thereof to further increase sensitivity, specificity, and predictive value of the assay.

### Example 5

### Multiplexing Protein and Nucleic Biomarkers on a Single Analytical Platform

MMP2 protein levels and FGFR3 mutations were detected on a single qPCR platform for a multi-analyte screening assay. To assay both MMP2 and FGFR3 simultaneously on a single analytical platform, six DNA aptamers tagged with unique florescence probes were designed to specifically bind to MMP2. Once bound to the MMP2 protein, these aptamers were utilized as templates for quantitative PCR mediated protein detection.

For sample preparation of MMP2, MMP2 protein was bound to the one of the six DNA aptamers in solution. The protein/aptamer complexes were then immunoprecipitated using anti-MMP2 specific antibodies. The MMP2-aptamer complexes were eluted with IgG elution buffer and neutralized with neutralizing buffer. The eluates were then used as the template in the multiplex qPCR reaction to detect the amount of the aptamers, and thereby detect the amount of MMP2 in the sample. For nucleic acid sample preparation, a PCR-clamping methodology was utilized on human genomic DNA designed to detect FGFR3 mutations. Wild-type blocking oligonucleotides containing locked nucleic acid (LNA) bases surrounding known mutation sites were included along with real-time PCR primers and duel-labeled taqman probes. The eluates (tagged aptamer-bound MMP2 plus immunoprecipitation) were added to the tagged nucleic acid sample pool and multiplex qPCR was carried out. Both the aptamers and the FGFR3 were detected in the multiplex qPCR. Accordingly, this method exemplifies a non-invasive assay in which proteins and nucleic acids can be simultaneously detected using a single analytical platform. In addition, the described methods are not limited the qPCR detection method or the specific proteins and nucleic acids, rather any detection method is suitable for multi-plex detection of any proteins and/or nucleic acids.

APPENDIX A

| | | Seq | |
|---|---|---|---|
| | | ID | |
| Assay | Reagent | No. | Sequence |
| FGFR3 | Exon 7 | 1 | 5′ GCG GTC CCA AAA GGG TCA GTA CAG TGG CGG TGG TGG |
| primary PCR | forward | | TGA GGG AG 3′ |
| | Exon 7 | 2 | 5′ GCG GTC CCA AAA GGG TCA GTA CGC ACC GCC GTC TGG |
| | reverse | | TTG G 3′ |
| | Exon 10 | 3 | 5′ GCG GTC CCA AAA GGG TCA GTA CGG TCT GGC CCT CTA |
| | forward | | GAC TCA 3′ |
| | Exon 10 | 3 | 5′ GCG GTC CCA AAA GGG TCA GTA CGG TCT GGC CCT CTA |
| | reverse | | GAC TCA 3′ |
| | Exon 15 | 4 | 5′ GCG GTC CCA AAA GGG TCA GTA CCC TGC CCT GAG ATG |
| | forward | | CT 3′ |
| | Exon 15 | 5 | 5′ GCG GTC CCA AAA GGG TCA GTA CCG TCC TAC TGG CAT |
| | reverse | | GAC C 3′ |
| FGFR3 mutation | Exon 7 | 6 | 5′ GCG TCA TCT GCC CCC A 3′ |
| detection | forward | | |
| | Exon 7 | 7 | 5′ CAC CGC CGT CTG GTT G 3′ |
| | reverse | | |
| | Exon 7 | 8 | 5′ AGA GCG CTC CCC G 3′ |
| | LNA | | |
| | Exon 7 | 9 | 5′ FAM-CCC GCC TGC AGG ATG GGC CGG T-Iowa black |
| | probe | | FQ 3′ |
| | Exon 10 | 10 | 5′ GGC CTC AAC GCC CAT GT 3′ |
| | forward | | |
| | Exon 10A | 11 | 5′ TAG CTG AGG ATG CCT GCA TA 3′ |
| | reverse | | |
| | Exon 10B | 12 | 5′ CCG TAG CTG AGG ATG CCT G 3′ |
| | reverse | | |
| | Exon 10A | 13 | 5′ ATA CAC ACT GCC CGC CT 3′ |
| | LNA | | |
| | Exon 10B | 14 | 5′ GCC TGC ATA CAC ACT 3′ |
| | LNA | | |
| | Exon 10 | 15 | 5′ FAM-CCG AGG AGG AGC TGG TGG AGG CTG AC-Iowa |
| | probe | | black FQ 3′ |
| | Exon 15 | 16 | 5′ CAA TGT GCT GGT GAC CGA G 3′ |
| | forward | | |
| | Exon 15 | 17 | 5′ CCG GGC TCA CGT TGG TC 3′ |
| | reverse | | |
| | Exon 15 | 18 | 5′ GGT CGT CTT CTT GTA GT 3′ |
| | LNA | | |
| | Exon 15 | 19 | 5′ FAM-CTG GCC CGG GAC GTG CAC AAC CTC GAC |
| | probe | | T-Iowa black FQ 3′ |
| Twist/Nid | Twist | 20 | 5′ GTT AGG GTT CGG GGG CGT TGT T 3′ |
| | forward | | |
| | Twist | 21 | 5′ CCG TCG CCT TCC TCC GAC GAA 3′ |
| | reverse | | |
| | Nid | 22 | 5′ GCG GTT TTT AAG GAG TTT TAT TTT C 3′ |
| | forward | | |
| | Nid | 23 | 5′ CTA CGA AAT TCC CTT TAC GCT 3′ |
| | reverse | | |
| ACTB | ACTB | 24 | 5′ TAG GGA GTA TAT AGG TTG GGG AAG TT 3′ |
| | forward | | |
| | ACTB | 25 | 5′ AAC ACA CAA TAA CAA ACA CAA ATT CAC 3′ |
| | reverse | | |
| | ACTB zen | 26 | 5′ TGG GGT GGT/ZEN/GAT GGA GGA GGT TTA GTA AGT |
| | probe | | TTT TT 3′ |

Abbreviations: ACTB, Actin-β: PCR, polymerise chain reaction.

What is claimed is:

1. A method of screening for cancer, the method comprising:
identifying a threshold parameter of MMP2 or MMP9 protein and of two or more nucleic acids selected from the group consisting of FGFR3, p53, TWIST1, Vimentin, and NID2, wherein said threshold parameters are indicative of the absence of cancer;
conducting an assay in a tissue or body fluid sample in order to determine a parameter of two or more nucleic acids selected from the group consisting of nucleic acid encoding FGFR3, p53, TWIST1, Vimentin, and NID2;
determining a parameter of MMP2 or MMP9 protein in said sample;
identifying said sample as positive for cancer if the parameters of at least one of said nucleic acids and the parameter of said protein present in said sample are greater than their respective threshold parameters.

2. The method of claim 1, wherein the cancer is a bladder cancer.

3. The method of claim 1, wherein the sample is selected from urine or blood.

4. The method of claim 1, wherein the nucleic acid is DNA or RNA.

5. The method of claim 1, wherein the parameter comprises a methylation pattern in the one or more of said nucleic acids.

6. The method of claim 1, wherein the parameter comprises a mutation in at least one of said nucleic acids.

7. The method of claim 6, wherein said mutation is selected from a loss of heterozygosity, a single nucleotide polymorphism, a deletion, an insertion, a rearrangement, and a translocation.

8. The method of claim 1, wherein the parameter comprises a level of protein expression of said protein.

9. The method of claim 1, wherein the parameter comprises a level of gene expression of at least one of said nucleic acids.

10. The method of claim 1, wherein said assay comprises sequencing said nucleic acid.

11. The method of claim 1, wherein said conducting and determining steps comprise
obtaining a sample comprising two or more said nucleic acids and MMP-2 or MMP-9 protein;
introducing an aptamer that binds to MMP-2 or MMP-9 protein in the sample;
removing unbound aptamer; and
conducting a single assay, wherein the assay detects both said nucleic acids and said protein, the assay comprising: performing a sequencing reaction on the two or more said nucleic acid and the aptamer, thereby detecting the nucleic acid and the aptamer in the sample.

12. The method of claim 11, wherein said assay is a single molecule assay.

13. The method of claim 12, wherein said single molecule assay is an ion semiconductor sequencing assay.


SEQUENCE LISTING (26 sequences)

SEQ ID NO: 1 - DNA, 44 bp, Artificial Sequence
  Synthetic Sequence
gcggtcccaaaagggtcagtacagtggcggtggtggtgagggag

SEQ ID NO: 2 - DNA, 40 bp, Artificial Sequence
  Synthetic Sequence
gcggtcccaaaagggtcagtacgcaccgccgtctggttgg

SEQ ID NO: 3 - DNA, 42 bp, Artificial Sequence
  Synthetic Sequence
gcggtcccaaaagggtcagtacggtctggccctctagactca

SEQ ID NO: 4 - DNA, 38 bp, Artificial Sequence
  Synthetic Sequence
gcggtcccaaaagggtcagtaccctgccctgagatgct

SEQ ID NO: 5 - DNA, 40 bp, Artificial Sequence
  Synthetic Sequence
gcggtcccaaaagggtcagtaccgtcctactggcatgacc

SEQ ID NO: 6 - DNA, 16 bp, Artificial Sequence
  Synthetic Sequence
gcgtcatctgccccca

SEQ ID NO: 7 - DNA, 16 bp, Artificial Sequence
  Synthetic Sequence
caccgccgtctggttg

SEQ ID NO: 8 - DNA, 13 bp, Artificial Sequence
  Synthetic Sequence
agagcgctccccg

SEQ ID NO: 9 - DNA, 22 bp, Artificial Sequence
  Synthetic Sequence
cccgcctgcaggatgggccggt

SEQ ID NO: 10 - DNA, 17 bp, Artificial Sequence
  Synthetic Sequence
ggcctcaacgcccatgt

SEQ ID NO: 11 - DNA, 20 bp, Artificial Sequence
  Synthetic Sequence
tagctgaggatgcctgcata

SEQ ID NO: 12 - DNA, 19 bp, Artificial Sequence
  Synthetic Sequence
ccgtagctgaggatgcctg

SEQ ID NO: 13 - DNA, 17 bp, Artificial Sequence
  Synthetic Sequence
atacacactgcccgcct

SEQ ID NO: 14 - DNA, 15 bp, Artificial Sequence
  Synthetic Sequence
gcctgcatacacact

SEQ ID NO: 15 - DNA, 26 bp, Artificial Sequence
  Synthetic Sequence
ccgaggaggagctggtggaggctgac

SEQ ID NO: 16 - DNA, 19 bp, Artificial Sequence
  Synthetic Sequence
caatgtgctggtgaccgag

SEQ ID NO: 17 - DNA, 17 bp, Artificial Sequence
  Synthetic Sequence
ccgggctcacgttggtc

SEQ ID NO: 18 - DNA, 17 bp, Artificial Sequence
  Synthetic Sequence
ggtcgtcttcttgtagt

SEQ ID NO: 19 - DNA, 28 bp, Artificial Sequence
  Synthetic Sequence
ctggcccgggacgtgcacaacctcgact

SEQ ID NO: 20 - DNA, 22 bp, Artificial Sequence
  Synthetic Sequence
gttagggttcgggggcgttgtt

SEQ ID NO: 21 - DNA, 21 bp, Artificial Sequence
  Synthetic Sequence
ccgtcgccttcctccgacgaa

SEQ ID NO: 22 - DNA, 25 bp, Artificial Sequence
  Synthetic Sequence
gcggtttttaaggagttttattttc

SEQ ID NO: 23 - DNA, 21 bp, Artificial Sequence
  Synthetic Sequence
ctacgaaattccctttacgct

SEQ ID NO: 24 - DNA, 26 bp, Artificial Sequence
  Synthetic Sequence
tagggagtatataggttggggaagtt

SEQ ID NO: 25 - DNA, 27 bp, Artificial Sequence
  Synthetic Sequence
aacacacaataacaaacacaaattcac

SEQ ID NO: 26 - DNA, 35 bp, Artificial Sequence
  Synthetic Sequence
tggggtggtgatggaggaggtttagtaagtttttt